(19) United States
(12) Patent Application Publication
(10) Pub. No.: US 2023/0096137 A1
(43) Pub. Date: Mar. 30, 2023

(54) COMPOSITION FOR LITHOGRAPHY AND PATTERN FORMATION METHOD

(71) Applicant: Mitsubishi Gas Chemical Company, Inc., Tokyo (JP)

(72) Inventors: Tadashi OMATSU, Hiratsuka-shi, Kanagawa (JP); Masahiro MATSUMOTO, Niigata-shi, Niigata (JP); Takashi SATO, Hiratsuka-shi, Kanagawa (JP); Masatoshi ECHIGO, Chiyoda-ku, Tokyo (JP)

(21) Appl. No.: 17/797,803

(22) PCT Filed: Feb. 2, 2021
(86) PCT No.: PCT/JP2021/003658

§ 371 Date: Aug. 5, 2022

(30) Foreign Application Priority Data
Feb. 6, 2020 (JP) 2020-018611
May 27, 2020 (JP) 2020-092060

(51) Int. Cl. G03F 7/11; C07C 39/15; C07C 39/367; C07D 311/78; C07D 207/20; C08F 212/14; C08F 220/14; C09D 125/18; C09D 133/12; C08F 220/22; C09D 133/16; C08F 220/18; C09D 133/08

(52) U.S. Cl. CPC G03F 7/11; C07C 39/15; C07C 39/367; C07D 311/78; C07D 207/20; C08F 212/24; C08F 220/14; C09D 125/18; C09D 133/12; C08F 220/22; C09D 133/16; C08F 220/1811; C09D 133/08

(57) ABSTRACT

An object of the present invention is to provide a composition for lithography capable of obtaining a film in contact with a resist layer or an underlayer film capable of forming a pattern excellent in exposure sensitivity the like. The object can be achieved by a composition for lithography containing a compound having at least one element selected from the group consisting of iodine, tellurium, and fluorine, or a resin having a constituent unit derived from the compound, wherein a total mass of the atoms in the compound is 15% by mass or more and 75% by mass or less.

### TECHNICAL FIELD

The present invention relates to a composition for lithography and a pattern formation method.

### BACKGROUND ART

In recent years, in the production of semiconductor elements and liquid crystal display elements, semiconductors (patterns) and pixels have been rapidly miniaturized due to the advance in lithography technology. As an approach for pixel miniaturization, the exposure light source has been shifted to have a shorter wavelength, in general. Specifically, ultraviolet rays typified by g-ray and i-ray have been used conventionally, but nowadays, far ultraviolet exposure such as KrF excimer laser (248 nm) and ArF excimer laser (193 nm) is being the center of mass production. Furthermore, the introduction of extreme ultraviolet (EUV) lithography (13.5 nm) is progressing. In addition, electron beam (EB) is also used for forming a fine pattern.

In particular, lithography using extreme ultraviolet rays has been increasingly introduced due to recent technical progress.

Up to now, typical resist materials are polymer based resist materials capable of forming an amorphous film. Examples include polymer based resist materials such as polymethyl methacrylate, polyhydroxy styrene with an acid dissociation group, and polyalkyl methacrylate (see, for example, Non Patent Literature 1).

Conventionally, a line pattern of about 10 to 100 nm is formed by irradiating a resist thin film made by coating a substrate with a solution of these resist materials with ultraviolet, far ultraviolet, electron beam, extreme ultraviolet or the like.

In addition, lithography using electron beam or extreme ultraviolet has a reaction mechanism different from that of normal photolithography (Non Patent Literatures 2 and 3). Furthermore, lithography with electron beam or extreme ultraviolet aims at forming fine patterns of several nm to ten-odd nm. Accordingly, there is a demand for a resist composition having higher sensitivity for an exposing source when the resist pattern dimension is reduced. In particular, lithography with extreme ultraviolet is required to further increase sensitivity in terms of throughput.

As a resist material that solves the problems as mentioned above, an inorganic resist material having a metallic element such as titanium, tin, hafnium and zirconium has been proposed (see, for example, Patent Literature 1).

### CITATION LIST

### Patent Literature

Patent Literature 1: Japanese Patent Laid-Open No. 2015-108781

### Non Patent Literature

Non Patent Literature 1: Shinji Okazaki et al., “Development of Lithography Technology in These 40 Years”, S&T Publishing Inc., Dec. 9, 2016
Non Patent Literature 2: H. Yamamoto, et al., Jpn. J. Appl. Phys. 46, L142 (2007)
Non Patent Literature 3: H. Yamamoto, et al., J. Vac. Sci. Technol. b 23, 2728 (2005)

### SUMMARY OF INVENTION

### Technical Problem

However, conventionally developed resist compositions having highly sensitive features have problems such as insufficient pattern quality due to large pattern defects and roughness, insufficient improvement in sensitivity, and insufficient etching resistance. In view of these circumstances, there is a need for a lithography technique that achieves both high resolution and high sensitivity.

Further, in lithography using extreme ultraviolet rays, since wavelengths as short as 13.5 nm are used, compared with conventional exposure techniques, the transmittance of photons is high and the number of photons at the same exposure intensity is small, and therefore, it is necessary to efficiently convert extreme ultraviolet rays into protons necessary for exposure. Furthermore, it is necessary to supply protons also from a layer adjacent to the resist.

In view of the above circumstances, an object of the present invention is to provide a composition for lithography capable of obtaining a film in contact with a resist layer (hereinafter referred to as a “resist layer contact film”) or an underlayer film capable of forming a pattern excellent in exposure sensitivity, and a pattern formation method.

### Solution to Problem

As a result of intensive studies to solve the problems described above, the present inventors have found that the exposure sensitivity in a lithography step can be increased by using a compound having a specific elemental composition or a resin containing the compound as a structural unit in a resist layer contact film or an underlayer film, thereby completing the present invention.

More specifically, the present invention is as follows.

[1]

A composition for lithography comprising a compound having at least one element selected from the group consisting of iodine, tellurium and fluorine or a resin having a constituent unit derived from the compound,

wherein a total mass of the elements in the compound is 15% by mass or more and 75% by mass or less.

[2]

The composition for lithography according to [1], wherein the at least one element is at least one element selected from the group consisting of iodine and tellurium.

[3]

The composition for lithography according to [1] or [2], wherein the at least one element is iodine, and a mass of the iodine in the compound is 15% by mass or more and 75% by mass or less.

[4]

The composition for lithography according to any one of [1] to [3], wherein the compound is represented by formula (A-4a):

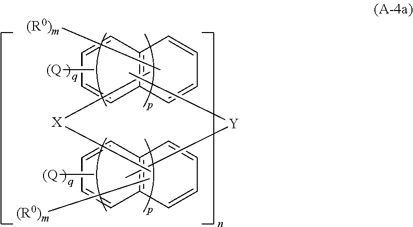

wherein

X represents an oxygen atom, a sulfur atom, a single bond, or not a crosslink;

Y is a 2n-valent group having 1 to 60 carbon atoms or a single bond;

wherein when X is not a crosslink, Y is the 2n-valent group;

each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;

where at least one R⁰ is a hydroxy group;

each m is independently an integer of 1 to 9;

Q represents iodine, tellurium, fluorine, an alkyl group having 1 to 30 carbon atoms and containing at least iodine, tellurium or fluorine, or an aryl group having 6 to 40 carbon atoms and containing at least iodine, tellurium or fluorine;

n is an integer of 1 to 4;

each p is independently an integer of 0 to 3;

at least one of Q, R⁰ and Y contains at least one element selected from the group consisting of iodine, tellurium, and fluorine; and

each q is independently an integer of 0 to (4+2×p−m).

[4-1]

The composition for lithography according to [4], wherein X is an oxygen atom or not a crosslink.

[4-2]

The composition for lithography according to [4] or [4-1], wherein Q is iodine.

[4-3]

The composition for lithography according to any one of [4] to [4-2], wherein at least one of Q, R⁰ and Y contains iodine.

[4-4]

The composition for lithography according to any one of [4] to [4-3], wherein each q is independently an integer of 1 to (4+2×p−m).

[5]

The composition for lithography according to any one of [4] to [4-4], wherein Y is a 2n-valent hydrocarbon group having an aryl group having 6 to 60 carbon atoms and optionally having a substituent.

[5-1]

The composition for lithography according to [5], wherein the 2n-valent hydrocarbon group is a methylene group.

[5-2]

The composition for lithography according to [5] or [5-1], wherein the aryl group having 6 to 60 carbon atoms is a phenyl group or a biphenyl group.

[5-3]

The composition for lithography according to any one of [5] to [5-2], wherein the substituent is iodine.

[6]

The composition for lithography according to any one of [1] to [3], wherein the compound is represented by formula (A-4c):

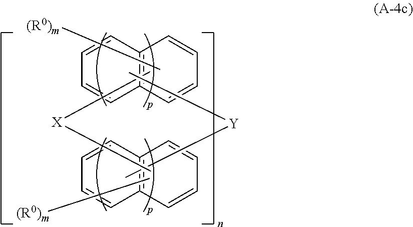

wherein

X represents an oxygen atom, a sulfur atom, a single bond, or not a crosslink;

Y is a 2n-valent group having 1 to 60 carbon atoms or a single bond;

wherein when X is not a crosslink, Y is the 2n-valent group;

each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;

where at least one R⁰ is a hydroxy group;

at least one R⁰ is iodide or an iodine-containing group;

each m is independently an integer of 1 to 9;

n is an integer of 1 to 4; and

each p is independently an integer of 0 to 3.

[6-1]

The composition for lithography according to [6], wherein X is an oxygen atom or not a crosslink.

[7]

The composition for lithography according to [6] or [6-1], wherein Y is a 2n-valent hydrocarbon group having an aryl group having 6 to 60 carbon atoms and optionally having a substituent.

[7-1]

The composition for lithography according to [7], wherein the 2n-valent hydrocarbon group is a methylene group.

[7-2]

The composition for lithography according to [7] or [7-1], wherein the aryl group having 6 to 60 carbon atoms is a phenyl group or a biphenyl group.

[7-3]

The composition for lithography according to any one of [7] to [7-2], wherein the substituent is iodine.

[8]

The composition for lithography according to any one of [1] to [3], wherein the compound is represented by the general formula (AM1):

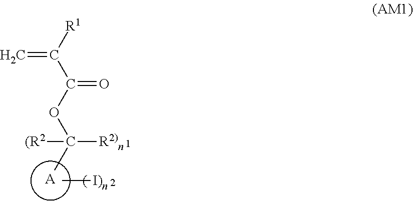

wherein

R¹ represents a hydrogen atom, methyl, or a halogen group;

each R² independently represents a hydrogen atom, a linear organic group having 1 to 20 carbon atoms, a branched organic group having 3 to 20 carbon atoms, or a cyclic organic group having 3 to 20 carbon atoms;

A represents an organic group having 1 to 30 carbon atoms;

n¹ represents 0 or 1; and

n² represents an integer of 1 to 20.

[8-1]

The composition for lithography according to [8], wherein R¹ is methyl.

[8-2]

The composition for lithography according to [8] or [8-1], wherein n¹ is 0.

[8-3]

The composition for lithography according to any one of [8] to [8-2], wherein A is an alicyclic hydrocarbon group.

[8-4]

The composition for lithography according to any one of [8] to [8-3], wherein A is an adamantyl group.

[9]

The composition for lithography according to any one of [1] to [3], wherein the compound is represented by the general formula (A-7):

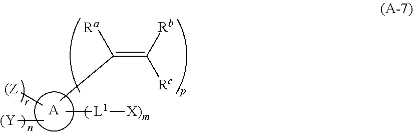

wherein

each X independently represents tellurium, I, F, or an organic group having 1 to 30 carbon atoms and having 1 or more and 5 or less substituents selected from the group consisting of tellurium, I, and F, and at least one X is tellurium or I;

L¹ represents a single bond, an ether group, an ester group, a thioether group, an amino group, a thioester group, an acetal group, a phosphine group, a phosphone group, a urethane group, a urea group, an amide group, an imide group, or a phosphate group;

m is an integer of 1 or more;

each Y independently represents a hydroxy group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group;

n is an integer of 0 or more;

each Z is independently an alkoxy group, an ester group, an acetal group, or a carbonate ester group;

r is an integer of 0 or more;

A is an organic group having 1 to 30 carbon atoms;

R^{a}, R^{b}, and R^{c} are each independently H, I, F, Cl, Br, or an organic group having 1 to 60 carbon atoms and optionally having a substituent; and

p is an integer of 1 or more.

[9-1]

The composition for lithography according to [9], wherein X is iodine.

[9-2]

The composition for lithography according to [9] or [9-1], wherein L¹ is a single bond.

[9-3]

The composition for lithography according to any one of [9] to [9-2], wherein Y is a hydroxy group, and n is an integer of 1 or more.

[9-4]

The composition for lithography according to any one of [9] to [9-3], wherein r is 0.

[9-5]

The composition for lithography according to any one of [9] to [9-4], wherein A is an aromatic ring having 6 to 14 carbon atoms.

[9-6]

The composition for lithography according to any one of [9] to [9-5], wherein R^{a}, R^{b}, and R^{c} are hydrogen.

[10]

The composition for lithography according to any one of [1] to [9-6], further comprising a solvent.

[11]

The composition for lithography according to any one of [1] to [10], further comprising an acid generating agent.

[12]

The composition for lithography according to any one of [1] to [11], further comprising an acid diffusion accelerator.

[13]

The composition for lithography according to any one of [1] to [12], further comprising an acid diffusion inhibitor.

[14]

The composition for lithography according to any one of [1] to [13], further comprising a crosslinking agent.

[15]

The composition for lithography according to any one of [1] to [14], wherein the composition is cured after formation of a thin film.

[16]

The composition for lithography according to any one of [1] to [15], wherein the composition is for forming a resist layer contact film.

[17]

The composition for lithography according to any one of [1] to [15], wherein the composition is for forming an underlayer film.

[18]

A method for forming a resist pattern, comprising:

an underlayer film formation step of forming an underlayer film on a substrate by using the composition for lithography according to [17];

a photoresist film formation step of forming at least one photoresist film on the underlayer film formed through the underlayer film formation step; and

a step of irradiating a predetermined region of the photoresist film formed through the photoresist film formation step with radiation for development.

[19]

A method for forming a circuit pattern, comprising:

an underlayer film formation step of forming an underlayer film on a substrate;

a resist layer contact film formation step of forming, by using the composition for lithography according to [16], a resist layer contact film on the underlayer film formed through the underlayer film formation step;

a photoresist film formation step of forming at least one photoresist film on the resist layer contact film formed through the resist layer contact film formation step;

a resist pattern formation step of irradiating a predetermined region of the photoresist film formed through the photoresist film formation step with radiation for development, thereby forming a resist pattern;

a pattern formation step of etching the resist layer contact film, or the resist layer contact film and the underlayer film with the resist pattern formed through the resist pattern formation step as a mask, thereby forming a pattern; and

a substrate pattern formation step of etching the substrate with the pattern formed through the pattern formation step as a mask, thereby forming a pattern on the substrate.

[20]

A compound represented by formula (A-4a):

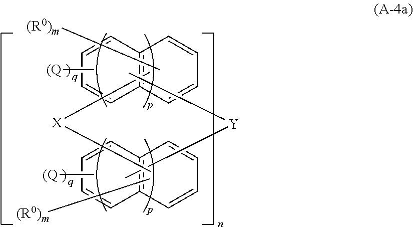

wherein

X represents an oxygen atom, a sulfur atom, a single bond, or not a crosslink;

Y is a 2n-valent group having 1 to 60 carbon atoms or a single bond;

wherein when X is not a crosslink, Y is the 2n-valent group;

each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;

where at least one R⁰ is a hydroxy group;

each m is independently an integer of 1 to 9;

Q represents iodine, tellurium, fluorine, an alkyl group having 1 to 30 carbon atoms and containing at least iodine, tellurium or fluorine, or an aryl group having 6 to 40 carbon atoms and containing at least iodine, tellurium or fluorine;

n is an integer of 1 to 4;

each p is independently an integer of 0 to 3;

at least one of Q, R⁰ and Y contains at least one element selected from the group consisting of iodine, tellurium, and fluorine; and

each q is independently an integer of 0 to (4+2×p−m).

[20-1]

The compound according to [20], wherein X is an oxygen atom or not a crosslink.

[20-2]

The compound according to [20] or [20-1], wherein Q is iodine.

[20-3]

The compound according to any one of [20] to [20-2], wherein at least one of Q, R⁰ and Y contains iodine.

[20-4]

The compound according to any one of [20] to [20-3], wherein each q is independently an integer of 1 to (4+2×p−m).

[21]

The compound according to any one of [20] to [20-4], wherein Y is a 2n-valent hydrocarbon group having an aryl group having 6 to 60 carbon atoms and optionally having a substituent.

[21-1]

The compound according to [21], wherein the 2n-valent hydrocarbon group is a methylene group.

[21-2]

The compound according to [21] or [21-1], wherein the aryl group having 6 to 60 carbon atoms is a phenyl group or a biphenyl group.

[21-3]

The compound according to any one of [21] to [21-2], wherein the substituent is iodine.

A compound represented by formula (A-4c):

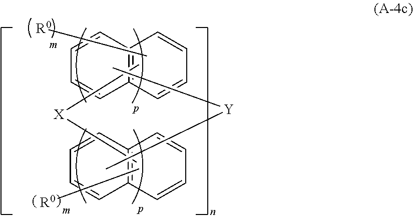

wherein

X represents an oxygen atom, a sulfur atom, a single bond, or not a crosslink;

Y is a 2n-valent group having 1 to 60 carbon atoms, or a single bond;

wherein when X is not a crosslink, Y is the 2n-valent group;

each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;

where at least one R⁰ is a hydroxy group;

at least one R⁰ is iodide or an iodine-containing group;

each m is independently an integer of 1 to 9;

n is an integer of 1 to 4; and

each p is independently an integer of 0 to 3.

[22-1]

The compound according to [22], wherein X is an oxygen atom or not a crosslink.

[23]

The compound according to [22] or [22-1], wherein Y is a 2n-valent hydrocarbon group having an aryl group having 6 to 60 carbon atoms and optionally having a substituent.

[23-1]

The compound according to [23], wherein the 2n-valent hydrocarbon group is a methylene group.

[23-2]

The compound according to [23] or [23-1], wherein the aryl group having 6 to 60 carbon atoms is a phenyl group or a biphenyl group.

[23-3]

The compound according to any one of [23] to [23-2], wherein the substituent is iodine.

### Advantageous Effects of Invention

According to the present invention, it is possible to provide a composition for lithography capable of increasing exposure sensitivity in a lithography step, and a pattern formation method.

### DESCRIPTION OF EMBODIMENTS

Hereinafter, an embodiment of the present invention will be described (hereinafter, may be referred to as the “present embodiment”). The present embodiment is given in order to illustrate the present invention. The present invention is not limited to only the present embodiment.

### [Compound (A) and Resin (A)]

A compound according to the present embodiment (hereinafter, also referred to as “compound (A)”) and a resin having a constituent unit derived from the compound (hereinafter, also referred to as “resin (A)”) contain at least one element selected from the group consisting of iodine, tellurium, and fluorine (preferably, the group consisting of iodine and tellurium). Since iodine and tellurium have a high ability to absorb extreme ultraviolet rays, they can absorb extreme ultraviolet rays to ionize the compound (A) and efficiently generate protons.

The total content of iodine and tellurium atoms is 15% by mass or more and 75% by mass or less of the entire compound (A), and preferably 20% by mass or more and 75% by mass or less.

When the total content of iodine and tellurium is 15% by mass or less, the ability to absorb extreme ultraviolet rays decreases, and thus the efficiency of proton generation decreases. When the total content of iodine and tellurium is 75% by mass or more, the stability of the compound decreases, and the compound is easily decomposed.

From the viewpoint of high density, the compound (A) preferably contains an aromatic ring. When the density is improved, the absorptance per passing length of the extreme ultraviolet rays is improved.

Further, the compound (A) preferably contains a hydrophilic group such as a hydroxy group from the viewpoint of adhesiveness to a substrate or a resist layer.

The “hydrophilic group” refers to a group that binds to an organic compound to improve the affinity between the organic compound and water. Examples of the hydrophilic group include a hydroxy group, a nitro group, an amino group, a carboxyl group, a thiol group, a phosphine group, a phosphone group, a phosphate group, an ether group, a thioether group, a urethane group, a urea group, an amide group, and an imide group.

The compound (A) preferably has curability and solvent resistance after curing so as to form a film and so as not to be dissolved in a resist solution when coated with a resist. Therefore, for example, the compound (A) preferably contains a crosslinkable group or a polymerizable group.

The “crosslinkable group” refers to a group that crosslinks in the presence of a catalyst or without a catalyst. Examples of the crosslinkable group include, but not particularly limited to, an alkoxy group having 1 to 20 carbon atoms, a group having an allyl group, a group having a (meth)acryloyl group, a group having an epoxy (meth)acryloyl group, a group having a hydroxy group, a group having a urethane (meth)acryloyl group, a group having a glycidyl group, and a group having a vinyl containing phenylmethyl group.

Further, the “polymerizable group” refers to a group that polymerizes in the presence of a catalyst or without a catalyst. Examples of the polymerizable group include, but not particularly limited to, a group having a (meth)acrylic group, a group having an unsaturated double bond such as a vinyl group, and a group having an unsaturated triple bond such as a propargyl group.

The compound (A) preferably contains a dissociation group.

The “dissociation group” refers to a group that is dissociated in the presence of a catalyst or without a catalyst. Among the dissociation groups, the acid dissociation group refers to a characteristic group that is cleaved in the presence of an acid and changes into an alkali soluble group or the like. Specific examples of the acid dissociation group include those described in International Publication No. WO 2016/158168. Preferable examples of the acid dissociation group include a group selected from the group consisting of a 1-substituted ethyl group, a 1-substituted n-propyl group, a 1-branched alkyl group, a silyl group, an acyl group, a 1-substituted alkoxymethyl group, a cyclic ether group, an alkoxycarbonyl group, and an alkoxycarbonylalkyl group, which has the property of being dissociated by an acid.

The resin (A) may be a resin obtained by independently polymerizing the compound (A), a resin polymerized by using a crosslinking agent, a resin copolymerized with another compound, or the like, and is not particularly limited.

The weight average molecular weight of the resin (A) is preferably 300 to 20000, more preferably 300 to 10000, and still more preferably 300 to 8000 from the viewpoints of reducing defects in a film to be formed and of a good pattern shape. As the above weight average molecular weight, a value obtained by measuring the weight average molecular weight in terms of polystyrene, using GPC, can be used.

For the production of the resin (A), any known method can be used without limitation as long as it is a method capable of producing a resin having the compound (A) as a constituent unit. Examples thereof include, but not particularly limited to, a method for crosslinking with an aldehyde, a ketone, a carboxylic acid, a carboxylic acid halide, a halogen-containing compound, an amino compound, an imino compound, an isocyanate or the like, and a method for copolymerizing with an unsaturated hydrocarbon group-containing compound or the like. Examples of the “unsaturated hydrocarbon group-containing compound” include, but not particularly limited to, a compound having a (meth)acrylic group, a compound having an unsaturated double bond such as a vinyl group, and a compound having an unsaturated triple bond such as a propargyl group.

The resin (A) can also be obtained in the synthetic reaction of the compound (A). For example, in synthesizing the compound (A), a method of obtaining the resin (A) from the raw material of the compound (A) may be employed.

The compound (A) is preferably a compound (A-1) represented by formula (A-1) containing a predetermined amount of one or more selected from the group consisting of iodine, tellurium, and fluorine.

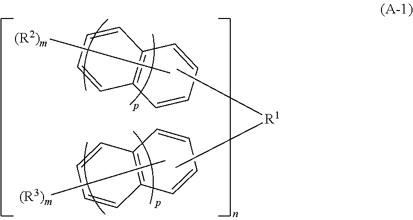

(In the general formula (A-1), each X independently represents an oxygen atom, a sulfur atom, or not a crosslink; R¹ is a single bond or a 2n-valent group having 1 to 30 carbon atoms; R² and R³ are each independently a halogen atom, a linear, branched, or cyclic alkyl group having 1 to 10 carbon atoms, an aryl group having 6 to 10 carbon atoms, an alkenyl group having 2 to 10 carbon atoms, an alkoxy group having 1 to 30 carbon atoms, a thiol group, or a hydroxy group; each m is independently an integer of 0 to 7, provided that at least one m is an integer of 1 to 7; each p is independently 0 or 1; and n is an integer of 1 to 4; provided that at least one selected from the group consisting of R¹, R², and R³ is a group containing one or more selected from the group consisting of an iodine atom, a tellurium atom, and a fluorine atom, and at least one R² and/or at least one R³ is one or more selected from a hydroxy group and a thiol group.)

The compound (A) is also preferably a compound (A-2) represented by formula (A-2) containing a predetermined amount of one or more selected from the group consisting of iodine, tellurium, and fluorine.

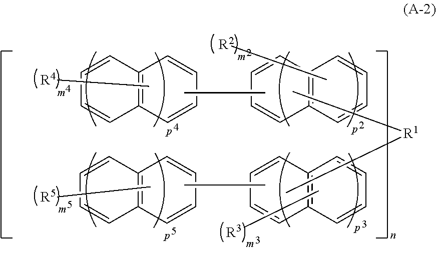

(In formula (A-2), R¹ is a 2n-valent group having 1 to 30 carbon atoms; R² to R⁵ are each independently a linear, branched, or cyclic alkyl group having 1 to 10 carbon atoms, an aryl group having 6 to 10 carbon atoms, an alkenyl group having 2 to 10 carbon atoms, an alkoxy group having 1 to 30 carbon atoms, a halogen atom, a thiol group, or a hydroxy group, provided that at least one selected from R¹ to R⁵ is a group containing one or more selected from the group consisting of an iodine atom, a tellurium atom, and a fluorine atom, and at least one R⁴ and/or at least one R⁵ is one or more selected from a hydroxy group and a thiol group; m² and m³ are each independently an integer of 0 to 8; m⁴ and m⁵ are each independently an integer of 0 to 9, provided that m⁴ and m⁵ are not 0 at the same time; n is an integer of 1 to 4; and p² to p⁵ are each independently an integer of 0 to 2.)

The compound (A) is also preferably a compound (A-3) represented by formula (A-3) containing a predetermined amount of one or more selected from the group consisting of iodine, tellurium, and fluorine.

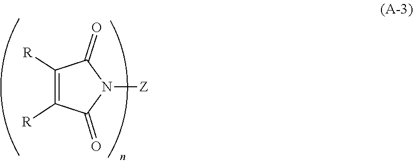

(In formula (A-3), each R is independently selected from the group consisting of a hydrogen atom, an alkyl group having 1 to 4 carbon atoms, and an aryl group having 6 to 12 carbon atoms; Z is an n-valent hydrocarbon group having 1 to 100 carbon atoms and optionally containing a heteroatom; n is 2 or more; and at least one selected from Z or R is a group containing one or more selected from the group consisting of an iodine atom, a tellurium atom, and a fluorine atom.)

The compound (A) is also preferably a compound (A-4a) or a compound (A-4b) represented by formula (A-4a) or formula (A-4b) containing a predetermined amount of one or more selected from the group consisting of iodine, tellurium, and fluorine. The resin having a constituent unit derived from the compound (A) of the present invention may be a polycyclic polyphenol resin having a constituent unit derived from the compound (A-4a) and/or the compound (A-4b). The polycyclic polyphenolic resin according to the present embodiment is a polycyclic polyphenolic resin having repeating units derived from at least one monomer selected from the group consisting of aromatic hydroxy compounds (A-4a) and (A-4b), wherein the repeating units are linked to each other by a direct bond between aromatic rings. Since the composition for film formation of the present embodiment is configured as described above, it has excellent film formability, heat resistance, and sublimation resistance.

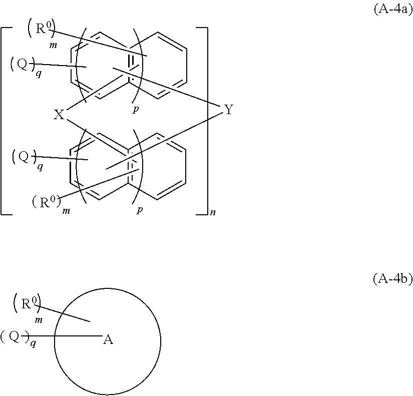

(In formula (A-4a), X represents an oxygen atom, a sulfur atom, a single bond, or not a crosslink, Y is a 2n-valent group having 1 to 60 carbon atoms, or a single bond, wherein when X is not a crosslink, Y is the 2n-valent group. Further, in formula (A-4b), A represents a benzene ring or a condensed ring. Furthermore, in the formulas (A-4a) and (A-4b), each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group, or a hydroxy group, wherein at least one R⁰ is a hydroxy group, and each m is independently an integer of 1 to 9. Q represents iodine, tellurium, fluorine, an alkyl group having 1 to 30 carbon atoms and containing at least iodine, tellurium or fluorine, or an aryl group having 6 to 40 carbon atoms and containing at least iodine, tellurium or fluorine. n is an integer of 1 to 4, and each p is independently an integer of 0 to 3. At least any one of Q, R⁰, X and Y contains at least one element of iodine, tellurium, and fluorine. In formula (A-4a), each q is independently an integer of 0 to (4+2×p−m). In formula (A-4b), each q is independently an integer of 0 to (2+2×p−m) (here, p represents the number of condensed rings in the condensed ring structure in formula (A-4b)).

X in formula (A-4 a) is preferably an oxygen atom from the viewpoint of heat resistance and reactivity. Furthermore, p in formula (A-4a) is preferably 1 from the viewpoint of heat resistance and solubility. Still further, in formula (A-4a), it is preferable that at least one R⁰ contains an iodine atom from the viewpoint of reactivity, and it is more preferable that Y does not contain an iodine atom from the viewpoint of storage stability.

Formula (A-4a) is preferably formula (A-4c).

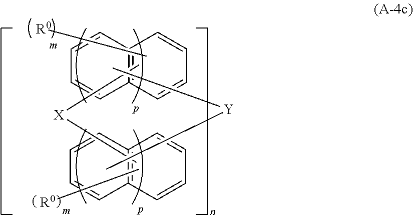

(In formula (A-4 c), X is an oxygen atom, a sulfur atom, a single bond, or not a crosslink, and Y is a 2n-valent group having 1 to 60 carbon atoms or a single bond, wherein when X is not a crosslink, Y is the 2n-valent group, and each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group, wherein at least one R⁰ is a hydroxy group and at least one R⁰ is an iodine atom or an iodine-containing group; each m is independently an integer of 1 to 9, n is an integer of 1 to 4, and p is each independently an integer of 0 to 3.)

### (Polycyclic Polyphenolic Resin)

The polycyclic polyphenolic resin according to the present embodiment typically has the following characteristics (1) to (5), but is not limited thereto.

(1) The polycyclic polyphenolic resin according to the present embodiment has excellent solubility in an organic solvent (particularly, a safe solvent). Therefore, for example, when the polycyclic polyphenolic resin according to the present embodiment is used as a film forming material for lithography, films for lithography can be formed by a wet process such as spin coating or screen printing.
(2) In the polycyclic polyphenolic resin according to the present embodiment, the carbon concentration is relatively high and the oxygen concentration is relatively low. In addition, since the polycyclic polyphenolic resin according to the present embodiment has a phenolic hydroxy group in the molecule, it is useful for formation of a cured product through the reaction with a curing agent, but it can also form a cured product on its own through the crosslinking reaction of the phenolic hydroxy group upon baking at a high temperature. Due to the above, the polycyclic polyphenolic resin according to the present embodiment can exhibit high heat resistance, and when used as a film forming material for lithography, degradation of the film upon baking at a high temperature is suppressed and a film for lithography excellent in etching resistance to oxygen plasma etching and the like can be formed.
(3) The polycyclic polyphenolic resin according to the present embodiment can exhibit high heat resistance and etching resistance, as described above, and also has excellent adhesiveness to a resist layer and a resist intermediate layer film material. Therefore, when the polycyclic polyphenolic resin according to the present embodiment is used as a film forming material for lithography, films for lithography excellent in resist pattern formability can be formed. The term “resist pattern formability” herein refers to a property in which there are no major defects in the resist pattern shape and both resolution and sensitivity are excellent.
(4) The polycyclic polyphenolic resin according to the present embodiment has high refractive index ascribable to its high aromatic ring density, and is prevented from being stained by heat treatment in a wide range from a low temperature to a high temperature, and is excellent in transparency, and therefore, the polycyclic polyphenolic resin according to the present embodiment is also useful as various optical component forming materials.
(5) The polycyclic polyphenol resin according to the present embodiment has Q as a functional group, the absorptance with respect to an EUV exposure light source can be improved, and in a case where the polycyclic polyphenol resin is used as an underlayer film for lithography, it is possible to lead to improvement in sensitivity and improvement in productivity by suppressing pattern defects such as pattern collapse.

It is considered that the polycyclic polyphenolic resin according to the present embodiment can be preferably applied as a film forming material for lithography due to such properties, and thus the above desired characteristics are imparted to the composition for film formation of the present embodiment. The composition for film formation of the present embodiment is not particularly limited as long as it contains the above polycyclic polyphenolic resin. That is, any optional component may be contained at any mixing ratio, and can be appropriately regulated according to a specific application of the composition for film formation.

Hereinafter, the formulas (A-4a) and (A-4b) described above will be described in detail.

In formula (A-4a), X represents an oxygen atom, a sulfur atom, a single bond or not a crosslink. X is preferably an oxygen atom from the viewpoint of heat resistance.

(In formula (A-4a), Y is a 2n-valent group having 1 to 60 carbon atoms, or a single bond, wherein when X is not a crosslink, Y is the 2n-valent group.

The 2n-valent group having 1 to 60 carbon atoms refers to, for example, a 2n-valent hydrocarbon group, and the hydrocarbon group may have various functional groups described later as substituents. Further, the 2n-valent hydrocarbon group refers to an alkylene group having 1 to 60 carbon atoms when n is 1, an alkanetetrayl group having 1 to 60 carbon atoms when n is 2, an alkanehexayl group having 2 to 60 carbon atoms when n is 3, and an alkaneoctayl group having 3 to 60 carbon atoms when n is 4. Examples of the 2n-valent hydrocarbon group include, for example, a group in which a 2n+1 valent hydrocarbon group is bonded to a linear hydrocarbon group, a branched hydrocarbon group, or an alicyclic hydrocarbon group. Herein, the alicyclic hydrocarbon group also includes a bridged alicyclic hydrocarbon group.

Examples of the 2n+1-valent hydrocarbon group include, but are not limited to, a 3-valent methine group and an ethyne group.

Further, the 2n-valent hydrocarbon group may have a double bond, a heteroatom, and/or an aryl group having 6 to 59 carbon atoms. Further, Y may contain a group derived from a compound having a fluorene skeleton such as fluorene or benzofluorene, but as used herein, the term “aryl group” is used to refer to a group that does not contain a group derived from a compound having a fluorene skeleton such as fluorene or benzofluorene.

In the present embodiment, the 2n-valent group may contain a halogen group, a nitro group, an amino group, a hydroxy group, an alkoxy group, a thiol group, or an aryl group having 6 to 40 carbon atoms. Furthermore, the 2n-valent group may contain an ether bond, a ketone bond, an ester bond, or a double bond.

In the present embodiment, from the viewpoint of heat resistance, the 2n-valent group preferably includes a branched hydrocarbon group or an alicyclic hydrocarbon group rather than a linear hydrocarbon group, and more preferably includes an alicyclic hydrocarbon group. Further, in the present embodiment, it is particularly preferably that the 2n-valent group has an aryl group having 6 to 60 carbon atoms.

Examples of the linear hydrocarbon group and the branched hydrocarbon group which may be contained in the 2n-valent group as a substituent include, but are not particularly limited to, an unsubstituted methyl group, an ethyl group, a n-propyl group, an i-propyl group, a n-butyl group, an i-butyl group, a t-butyl group, a n-pentyl group, a n-hexyl group, a n-dodecyl group, and a barrel group.

Examples of an alicyclic hydrocarbon group and an aromatic group having 6 to 60 carbon atoms which may be contained in the 2n-valent group as a substituent include, but are not particularly limited to, an unsubstituted phenyl group, a naphthalene group, a biphenyl group, an anthracyl group, a pyrenyl group, a cyclohexyl group, a cyclododecyl group, a dicyclopentyl group, a tricyclodecyl group, an adamantyl group, a phenylene group, a naphthalenediyl group, a biphenyldiyl group, an anthracenediyl group, a pyrendiyl group, a cyclohexanediyl group, a cyclododecanediyl group, a dicyclopentanediyl group, a tricyclodecanediyl group, an adamantanediyl group, a benzenetriyl group, a naphthalenetriyl group, a biphenyltriyl group, an anthracenetriyl group, a pyrenetriyl group, a cyclohexanetriyl group, a cyclododecanetriyl group, a dicyclopentanetriyl group, a tricyclodecanetriyl group, an adamantanetriyl group, a benzenetetrayl group, a naphthalenetetrayl group, a biphenyltetrayl group, an anthracenetetrayl group, a pyrenetetrayl group, a cyclohexanetetrayl group, a cyclododecanetetrayl group, a dicyclopentanetetrayl group, a tricyclodecantetrayl group, and an adamantanetetrayl group.

Each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group. The alkyl group may be either linear, branched or cyclic.

Here, at least one R⁰ is a hydroxy group.

Examples of the alkyl group having 1 to 40 carbon atoms include, but are not limited to, a methyl group, an ethyl group, a n-propyl group, an i-propyl group, a n-butyl group, an i-butyl group, a t-butyl group, a n-pentyl group, a n-hexyl group, a n-dodecyl group, and a barrel group.

Examples of the aryl group having 6 to 40 carbon atoms include, but are not limited to, a phenyl group, a naphthalene group, a biphenyl group, an anthracyl group, a pyrenyl group, and a perylene group.

Examples of the alkenyl group having 2 to 40 carbon atoms include, but are not limited to, an ethynyl group, a propenyl group, a butynyl group, and a pentynyl group.

Examples of the alkynyl group having 2 to 40 carbon atoms include, but are not limited to, an acetylene group, and an ethynyl group.

Examples of the alkoxy group having 1 to 40 carbon atoms include, but are not limited to, a methoxy group, an ethoxy group, a propoxy group, a butoxy group, and a pentoxy group.

Each m is independently an integer of 1 to 9. From the viewpoint of solubility, the number is preferably 1 to 6, more preferably 1 to 4, and from the viewpoint of raw material availability, 1 is still more preferable.

n is an integer of 1 to 4. From the viewpoint of solubility, the number is preferably 1 to 2, and from the viewpoint of raw material availability, 1 is still more preferable.

Each p is independently an integer of 0 to 3. From the viewpoint of heat resistance, the number is preferably 1 to 2, and from the viewpoint of raw material availability, 1 is still more preferable.

In the present embodiment, as the aromatic hydroxy compound, those represented by any of the formulas (A-4a) and (A-4b) can be used alone, or two or more kinds thereof can be used together. In the present embodiment, from the viewpoint of achieving both solvent solubility and heat resistance, it is preferable to adopt the compound represented by formula (A-4a) as the aromatic hydroxy compound. Further, from the viewpoint of achieving both solvent solubility and heat resistance, it is also preferable to adopt the compound represented by formula (A-4b) as the aromatic hydroxy compound.

The compound (A) of the present embodiment may be an oligomer (A-5) having an aralkyl structure represented by formula (A-5) which contains a predetermined amount of one or more selected from iodine, tellurium, and fluorine.

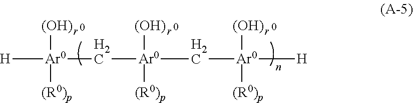

(wherein

Ar⁰ may be the same group or a different group, and each independently represents a divalent group containing a phenylene group, a naphthylene group, an anthrylene group, a phenanthrylene group, a pyrylene group, a fluorylene group, a biphenylene group, or a terphenylene group;

R⁰ may be the same group or a different group, and each independently represents an alkyl group having 1 to 30 carbon atoms and optionally having a substituent, an aryl group optionally having a substituent, iodine, tellurium, fluorine, or an alkyl group having 1 to 30 carbon atoms or an aryl group containing at least iodine, tellurium or fluorine;

n represents an integer of 1 to 50;

each r⁰ independently represents an integer of 0 to 3; and

each p independently represents an integer of 0 or more. However, not all r⁰ are 0 at the same time. Further, not all p are 0 at the same time. Further, at least one R⁰ contains any of iodine, tellurium, and fluorine.)

In the oligomer (A-5), Ar⁰ represents a divalent group containing a phenylene group, a naphthylene group, an anthrylene group, a phenanthrylene group, a pyrylene group, a fluorylene group, a biphenylene group or a terphenylene group, and a divalent group containing a phenylene group, a naphthylene group, an anthrylene group or a pyrylene group is preferable. Each Ar⁰ may independently be the same group or a different group.

Specific examples of Ar⁰ include a 1,4-phenylene group, a 1,3-phenylene group, a 4,4′-biphenylene group, a 2,4′-biphenylene group, a 2,2′-biphenylene group, a 2,3′-biphenylene group, a 3,3′-biphenylene group, a 3,4′-biphenylene group, a 2,6-naphthylene group, a 1,5-naphthylene group, a 1,6-naphthylene group, a 1,8-naphthylene group, a 1,3-naphthylene group, a 1,4-naphthylene group, an anthrylene group, a phenanthrylene group, a pyrylene group, a fluorylene group, and a terphenylene group. Examples of Ar⁰ also include divalent groups in which a plurality of phenylene groups or the like are linked with an alkylene group or the like, such as divalent groups having a diphenylmethyl structure, a bisphenol structure, or a bis(hydroxyphenyl) diisopropylphenyl structure.

R⁰ is a substituent of Ar⁰, may be the same group or a different group, and each independently represents an alkyl group having 1 to 30 carbon atoms and optionally having a substituent or an aryl group optionally having a substituent. Specific examples of R⁰ include specific examples of R^{a} and R^{b}, which will be described later.

In the oligomer (A-5), n represents an integer of 1 to 50. n is preferably 3 to 40, still more preferably 3 to 30, and particularly preferably 3 to 20, from the viewpoint of planarization performance of the film.

In the oligomer (A-5), each r⁰ independently represents an integer of 0 to 3. However, not all r⁰ are 0 at the same time. r⁰ is preferably 1 to 3 from the viewpoint of improvement in curability and solubility.

In the oligomer (A-5), each p independently represents an integer of 0 or more. However, not all p are 0 at the same time. p varies as appropriate depending on the type of Ar⁰.

The compound (A) is also preferably a compound (A-6) represented by formula (A-6) containing a predetermined amount of one or more selected from iodine, tellurium, and fluorine.

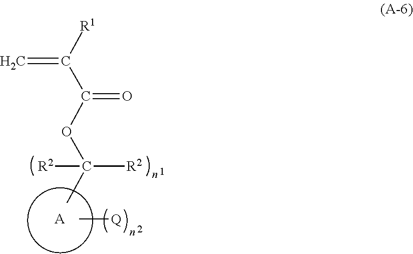

In formula (A-6),

R¹ represents a hydrogen atom or a methyl group or a halogen group;

each R² independently represents a hydrogen atom, a linear organic group having 1 to 20 carbon atoms, a branched organic group having 3 to 20 carbon atoms, or a cyclic organic group having 3 to 20 carbon atoms;

A represents an organic group having 1 to 30 carbon atoms;

each Q independently represents iodine, tellurium, fluorine, or an alkyl group having 1 to 30 carbon atoms and containing at least iodine, tellurium or fluorine or an aryl group containing at least iodine, tellurium or fluorine, and preferably Q is iodine;

n¹ represents 0 or 1; and

n² represents an integer of 1 to 20.

As R¹, a hydrogen atom or a methyl group or a halogen group can be used. As the halogen group, publicly known atoms can be used, and F, Cl, Br, I or the like can be appropriately used. R¹ is preferably a methyl group or a halogen group from the viewpoint of exposure sensitivity and material stability when the compound of the present invention is used as a constituent unit of resin for resists, more preferably a halogen group and still more preferably I, from the viewpoint of exposure sensitivity.

R² may be a combination of two or more selected from the group consisting of a linear organic group having 1 to 20 carbon atoms, a branched organic group having 3 to 20 carbon atoms, and a cyclic organic group having 3 to 20 carbon atoms.

R² is preferably a hydrogen atom for the purpose of suppressing an increase in Tg of the resin and improving the effect of introducing the iodine element. Further, for the purpose of controlling solubility in the developer, it is preferable to use an organic group having 1 or more carbon atoms in order to improve the acid decomposability. It is also preferable to use a hydrogen atom for the purpose of suppressing acid decomposability, especially ensuring solubility in an alkali developer and suppressing residue.

R² may have a substituent. Examples of R² include, for example, an alkyl group having 1 to 20, 1 to 10, or 1 to 6 carbon atoms, which may have a substituent; an alkenyl group having 2 to 20, 2 to 10, or 2 to 6 carbon atoms, which may have a substituent; an alkynyl group having 2 to 20, 2 to 10, or 2 to 6 carbon atoms, which may have a substituent; a cycloalkyl group having 3 to 20, 3 to 10, or 3 to 6 carbon atoms, which may have a substituent; a cycloalkenyl group having 3 to 20, 3 to 10, or 3 to 6 carbon atoms, which may have a substituent; a cycloalkynyl group having 3 to 20, 3 to 10, or 3 to 6 carbon atoms, which may have a substituent; an aryl group having 5 to 20, 5 to 10, or 5 to 6 carbon atoms, which may have a substituent; and combinations thereof.

Specific examples of R² include, for example, a methyl group, an ethyl group, a propyl group, a butyl group, a pentyl group, a hexyl group, a heptyl group, an octyl group, a nonyl group, a decyl group, an icosyl group, a cyclopropyl group, a cyclobutyl group, a cyclopentyl group, a cyclohexyl group, a cycloheptyl group, a cyclooctyl group, a cyclononyl group, a cyclodecyl group, a cycloicosyl group, an adamantyl group, an ethylene group, a propylene group, a butylene group, a phenyl group, a naphthyl group, an anthracene group, a phenanthrene group, a tetracene group, a chrysene group, a triphenylene group, a pyrene group, a benzopyrene group, an azulene group, and a fluorene group, which may have a substituent. These may each contain an ether bond, a ketone bond or an ester bond.

Here, the groups listed above include isomers. For example, the propyl group includes a n-propyl group and an isopropyl group, and the butyl group includes a n-butyl group, a sec-butyl group, an isobutyl group, and a tert-butyl group.

Examples of the substituent for R² include, but are not particularly limited to, a halogen atom, a hydroxy group, a cyano group, a nitro group, an amino group, a thiol group, a heterocyclic group, a linear aliphatic hydrocarbon group, a branched aliphatic hydrocarbon group, a cyclic aliphatic hydrocarbon group, an aryl group, an aralkyl group, an alkoxy group, an alkenyl group, an acyl group, an alkoxycarbonyl group, an alkyloyloxy group, an aryloyloxy group, an alkylsilyl group, and various crosslinkable groups and acid dissociation groups.

The “crosslinkable group” refers to a group capable of crosslinking by acid, alkali, light or heat in the presence of a catalyst or without a catalyst. Examples of the crosslinkable group include, but not particularly limited to, a group having an allyl group, a group having a (meth)acryloyl group, a group having an epoxy (meth)acryloyl group, a group having a urethane (meth)acryloyl group, a group having a hydroxy group, a group having a glycidyl group, a group having a vinyl containing phenylmethyl group, a group having a styrene group, a group having an alkynyl group, a group having a carbon-carbon double bond, a group having a carbon-carbon triple bond, and a group containing these groups.

The “acid dissociation group” is a group that is cleaved in the presence of an acid to generate an alkali soluble group (for example, a phenolic hydroxy group, a carboxyl group, a sulfonic acid group, or a hexafluoroisopropanol group) or the like. The acid dissociation group is not particularly limited, but can be arbitrarily selected for use from among, for example, those proposed in hydroxystyrene resins, (meth)acrylic acid resins, and the like for use in chemically amplified resist compositions for KrF or ArF. Specific examples of the acid dissociation group include, for example, those described in International Publication No. WO 2016/158168.

A may have a substituent. Examples of the compound serving as the skeleton of A include, for example, an alkane group having 1 to 30, 1 to 20, 1 to 10, or 1 to 6 carbon atoms, which may have a substituent; an alkene group having 2 to 30, 2 to 20, 2 to 10, or 2 to 6 carbon atoms, which may have a substituent; an alkyne group having 2 to 30, 2 to 20, 2 to 10, or 2 to 6 carbon atoms, which may have a substituent; a cycloalkane group having 3 to 30, 3 to 20, 3 to 10, or 3 to 6 carbon atoms, which may have a substituent; a cycloalkene group having 3 to 30, 3 to 20, 3 to 10, or 3 to 6 carbon atoms, which may have a substituent; a cycloalkyne group having 3 to 30, 3 to 20, 3 to 10, or 3 to 6 carbon atoms, which may have a substituent; an arene group having 5 to 30, 5 to 20, 5 to 10, or 5 to 6 carbon atoms, which may have a substituent; and combinations thereof.

Specific examples of the compound serving as the skeleton of A include, for example, methane, ethane, propane, butane, pentane, hexane, heptane, octane, nonane, decane, icosane, triacontane, cyclopropane, cyclobutane, cyclopentane, cyclohexane, cycloheptane, cyclooctane, cyclononane, cyclodecane, cycloicosane, cyclotriacontane, adamantane, ethylene, propylene, butene, pentene, hexene, heptene, octene, nonene, decene, icosene, triacontene, benzene, phenol, naphthalene, anthracene, phenanthrene, tetracene, chrysene, triphenylene, pyrene, pentacene, benzopyrene, coronene, azulene, and fluorene, which may have a substituent, and combinations thereof. These may each contain an ether bond, a ketone bond or an ester bond.

Examples of the substituent for the compound serving as the skeleton of A include, but are not particularly limited to, a halogen atom (fluorine, chlorine, bromine), a hydroxy group, a cyano group, a nitro group, an amino group, a thiol group, a heterocyclic group, a linear aliphatic hydrocarbon group, a branched aliphatic hydrocarbon group, a cyclic aliphatic hydrocarbon group, an aryl group, an aralkyl group, an alkoxy group, an alkenyl group, an acyl group, an alkoxycarbonyl group, an alkyloyloxy group, an aryloyloxy group, an alkylsilyl group, and various crosslinkable groups and acid dissociation groups.

The “crosslinkable group” and the “acid dissociation group” are not particularly limited, and for example, those described for R² can be used.

n¹ represents 0 or 1, and is preferably 1.

n² is an integer of 1 to 20, preferably an integer of 2 to 20, more preferably an integer of 2 to 10, and still more preferably an integer of 2 to 5.

The compound (A) according to the present embodiment may be a compound (A-7) represented by formula (A-7) which contains a predetermined amount of one or more selected from iodine, tellurium, and fluorine. The compound (A-7) preferably contains a functional group that improves solubility in an alkali developing solution by the action of an acid or a base. It is preferable that any one of Z, Y, and X described below contains a functional group that improves solubility in an alkali developing solution by the action of an acid or a base.

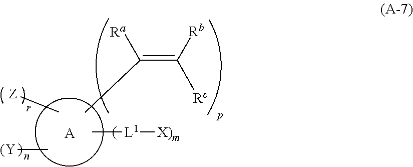

In formula (A-7),

each X independently represents tellurium, I, F, Cl, Br, or an organic group having 1 to 30 carbon atoms and having 1 or more and 5 or less substituents selected from the group consisting of tellurium, I, F, Cl, and Br. At least one X is tellurium or I.

L¹ represents a single bond, an ether group, an ester group, a thioether group, an amino group, a thioester group, an acetal group, a phosphine group, a phosphone group, a urethane group, a urea group, an amide group, an imide group, or a phosphate group. Among these, L1 is preferably a single bond.

m is an integer of 1 or more, preferably an integer of 1 or more and 5 or less, more preferably an integer of 2 or more and 4 or less, and still more preferably 2 or 3.

Each Y is independently a hydroxy group, an alkoxy group, an ester group, an acetal group, a carbonate group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, an ester group, a carbonate group, an amino group, an ether group, a thioether group, a phosphine group, a phosphone group, a urethane group, a urea group, an amide group, an imide group, and a phosphate group of Y may have a substituent.

Preferably, each Y is independently a group represented by the following formula (Y-1).

-L²-R² (Y-1)

In formula (Y-1),

L² is a group which is cleaved by the action of an acid. Examples of the group which is cleaved by the action of an acid include at least one divalent linking group selected from the group consisting of an ester group [*¹—O—(C═O)—*² or *¹—(C═O)—O—*²], an acetal group [[*¹—O—(C(R²¹)₂)—O—*²] (each R²¹ is independently H or a divalent hydrocarbon group having 1 to 10 carbon atoms), a carboxyalkoxy group [*¹—O—R²²—(C═O)—O—*²] (each R²² is independently H or a divalent hydrocarbon group having 1 to 10 carbon atoms), and a carbonate ester group [*¹—O—(C═O)—O—*²]. In the formula, *¹ represents a binding site to A, and *² represents a binding site to R². Among these, L² is preferably an acetal group, a carbonate ester group, or a carboxyalkoxy group.

R² is a linear, branched or cyclic aliphatic group having 1 to 30 carbon atoms, an aromatic group having 6 to 30 carbon atoms, a linear, branched or cyclic heteroatom-containing aliphatic group having 1 to 30 carbon atoms, or a heteroatom-containing aromatic group having 1 to 30 carbon atoms, and the aliphatic group, aromatic group, heteroatom-containing aliphatic group or heteroatom-containing aromatic group of R² may further have a substituent. Examples of the substituent herein include a linear, branched or cyclic aliphatic group having 1 to 20 carbon atoms and an aromatic group having 6 to 20 carbon atoms. Among these, R² is preferably an aliphatic group. The aliphatic group in R² is preferably a branched or cyclic aliphatic group. The aliphatic group preferably has 1 or more and 20 or less carbon atoms, more preferably 3 or more and 10 or less carbon atoms, and still more preferably 4 or more and 8 or less carbon atoms. Examples of the aliphatic group include, but are not particularly limited to, a methyl group, an isopropyl group, a sec-butyl group, a tert-butyl group, an isobutyl group, a cyclohexyl group, and a methylcyclohexyl group. Among these, a tert-butyl group or a cyclohexyl group is preferable.

When L² is *¹—(C═O)—O—*² or a carboxyalkoxy group, in a case where L² is cleaved by the action of an acid, a carboxylic acid group is formed, and the difference in solubility and the difference in dissolution rate between the disassembled portion and the undisassembled portion in the development process is increased, and therefore resolution is improved, and in particular, the residue at the bottom of the pattern in the fine wire pattern is suppressed, which is preferable.

Preferably, each Y is independently a group represented by any one of the following formulas (Y-1-1) to (Y-1-7).

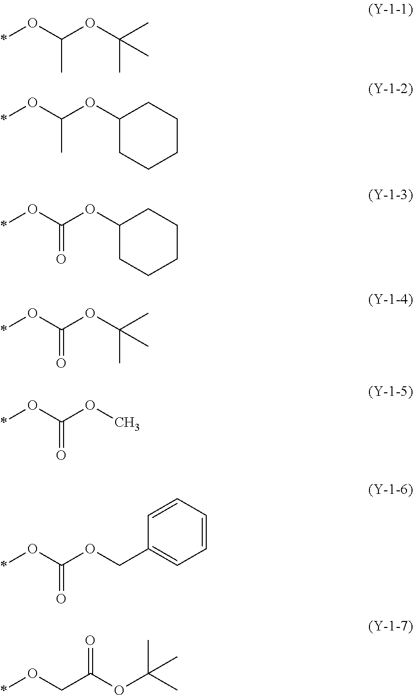

n is an integer of 0 or more, preferably an integer of 1 or more, more preferably an integer of 1 or more and 5 or less, still more preferably an integer of 1 or more and 3 or less, and yet still more preferably 1 or 2.

R^{a}, R^{b}, and R^{c} are each independently H, I, F, Cl, Br, or an organic group having 1 to 60 carbon atoms and optionally having a substituent. Examples of the substituent of the organic group having 1 to 60 carbon atoms include, but are not particularly limited to, I, F, Cl, Br, and other substituents. Examples of the other substituents include, but are not particularly limited to, a hydroxy group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, and a phosphate group. Among them, an alkoxy group, an ester group, a carbonate group, an amino group, an ether group, a thioether group, a phosphine group, a phosphone group, a urethane group, a urea group, an amide group, an imide group, and a phosphate group may further have substituents. Examples of the substituent herein include a linear, branched or cyclic aliphatic group having 1 to 20 carbon atoms and an aromatic group having 6 to 20 carbon atoms.

The organic group optionally having a substituent in R^{a}, R^{b}, and R^{c} preferably has 1 to 30 carbon atoms.

Examples of the organic group having 1 to 60 carbon atoms and optionally having a substituent include, but are not particularly limited to, a linear or branched aliphatic hydrocarbon group having 1 to 60 carbon atoms, an alicyclic hydrocarbon group having 4 to 60 carbon atoms, and an aromatic group having 6 to 60 carbon atoms and optionally containing a heteroatom.

Examples of the linear or branched aliphatic hydrocarbon group having 1 to 60 carbon atoms include, but are not particularly limited to, a methyl group, an ethyl group, a n-propyl group, an isopropyl group, a n-butyl group, an isobutyl group, a tert-butyl group, a n-pentyl group, a n-hexyl group, a n-dodecyl group, a barrel group, and a 2-ethylhexyl group.

Examples of the alicyclic hydrocarbon group include, but are not particularly limited to, a cyclohexyl group, a cyclododecyl group, a dicyclopentyl group, a tricyclodecyl group, and an adamantyl group. Furthermore, an aromatic group and optionally containing a heteroatom, such as a benzodiazole group, a benzotriazole group, or a benzothiadiazole group, can be appropriately selected. Combinations of these organic groups can also be selected.

Examples of the aromatic group having 6 to 60 carbon atoms and optionally containing a heteroatom include, but are not particularly limited to, a phenyl group, a naphthalene group, a biphenyl group, an anthracyl group, a pyrenyl group, a benzodiazole group, a benzotriazole group, and a benzothiadiazole group.

Among these organic groups having 1 to 60 carbon atoms and optionally having a substituent, a methyl group is preferable.

A is an organic group having 1 to 30 carbon atoms. A may be a monocyclic organic group or a polycyclic organic group. A is preferably an aromatic ring. A preferably has 6 to 14 carbon atoms, and more preferably 6 to 10 carbon atoms.

A is preferably a group represented by any one of the following formulae (A-1) to (A-4), and more preferably a group represented by the following formula (A-1).

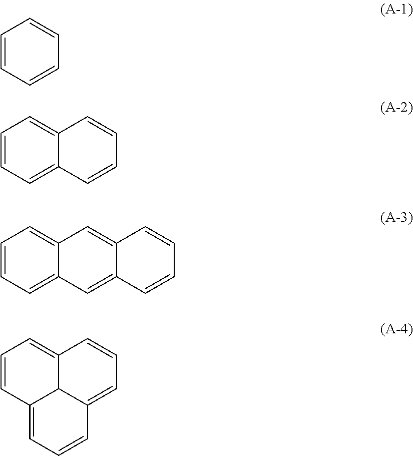

p represents the number of vinyl groups, and p is an integer of 1 or more, preferably an integer of 1 or more and 3 or less, more preferably an integer of 1 or more and 2 or less, and still more preferably 1.

Each Z is independently an alkoxy group, an ester group, an acetal group, or a carbonate ester group. r is an integer of 0 or more, preferably an integer of 0 or more and 2 or less, more preferably an integer of 0 or more and 1 or less, and still more preferably 0.

The resin having a constituent unit derived from the compound (A) of the present embodiment may have a constituent unit represented by the following formula (A-8). A resist composition containing the resin component can achieve high sensitivity in a lithography process and high resolution by increasing the solubility contrast of the resin in development.

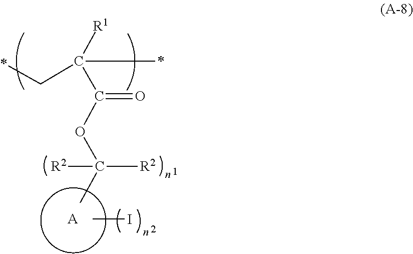

In formula (A-8), R¹, R², A, n¹, and n² are as defined in formula (A-6), and symbol * represents a bonding site to an adjacent repeating unit.

The (meth)acrylate (co)polymer represented by formula (A-8) can be obtained by polymerizing one or more (meth)acrylate compounds represented by formula (A-6), or by polymerizing one or more (meth)acrylate compounds represented by formula (A-6) with other monomers. The (meth)acrylate (co)polymer can be used as a material for film formation for lithography.

When the compound (A) and the resin derived from the compound (A) of the present embodiment are used as a resist underlayer film in an exposure process, in a case where a process for forming a layer to be processed by laminating a layer to be processed on a layer to be processed in a process step such as dry etching after pattern formation is used as an underlayer film of the resist layer, it is preferable to have a high carbon content, a low hydrogen content, and a high ring structure introduction rate so as to suppress an etching rate from the viewpoint of etching mask performance for improving pattern quality such as rectangularity and roughness after processing of the layer to be processed, and it is preferable to use a resin having a condensed ring structure in which a single ring or two or more ring structures are condensed as the compound structure. In this case, the ring structure preferably has an aromatic structure or a heteroaromatic structure.

In a case where the compound (A) and the resin derived from the compound (A) of the present embodiment are used as a resist underlayer film, and further one or more other spin-on-carbon (SOC) layers or inorganic hard mask layers are used as the etching mask layer, from the viewpoint of the processability of the etching mask layer, which is the target of etching the pattern shape of the resist layer, and of not degrading the pattern shape by etching from the shape of the resist immediately after development, it is also preferable to improve the quality of the pattern shape of the processed layer after processing by using a resist underlayer film made of the compound (A) or the resin derived from the compound (A) of the present embodiment, which shows faster etching rate and easier etching property than resist, and by laminating at least one or more etching mask layers on the substrate with the processed layer and further laminating a layer made of the compound (A) or the resin derived from the compound (A) of the present embodiment on the upper layer side than the etching mask layers. From these viewpoints, a resin having a small aromatic ring structure or a structure having no novolac structure is preferable, and a resin having an aliphatic structure in a main chain such as a polyacrylic resin, a polyethylene resin, a polyalkylene ether resin or the like or a resin having a high content ratio of a carbon skeleton constituting the aliphatic structure is preferable.

Specific examples of the compound (A) are shown below, but the compound (A) is not limited thereto:

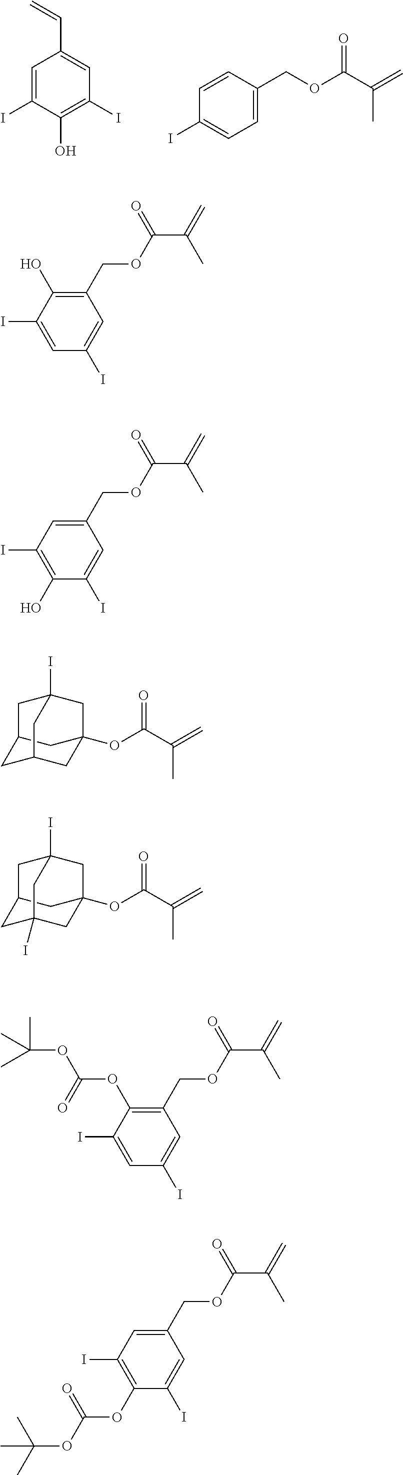

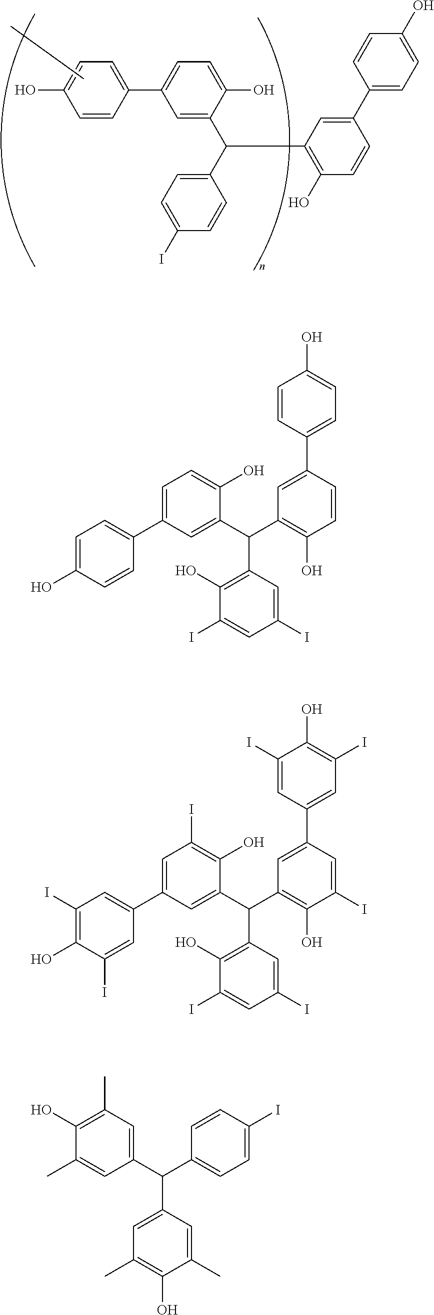

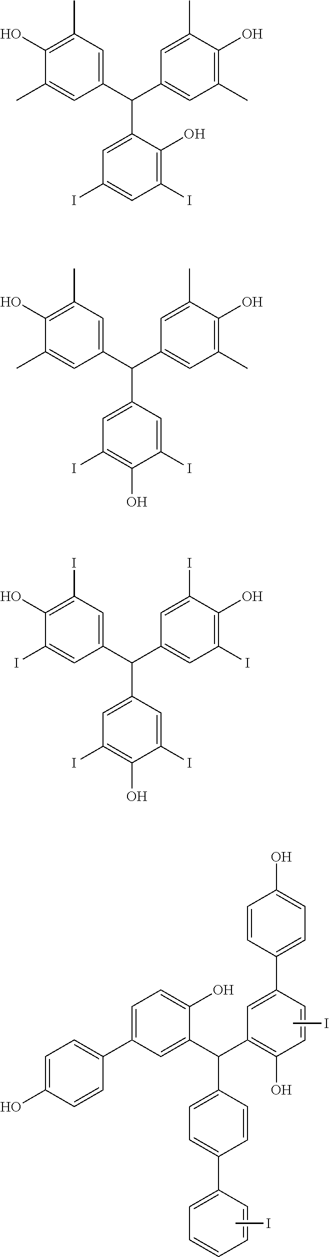

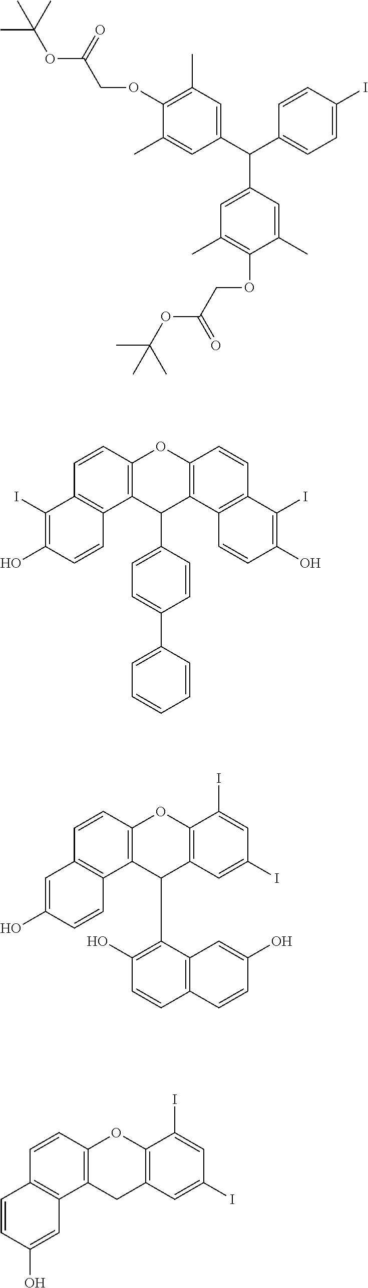 [SMILES: Cc1cc(C(c2ccc(I)cc2)c2cc(C)c(OCC(=O)OC(C)(C)C)c(C)c2)cc(C)c1OCC(=O)OC(C)(C)C ; Oc1ccc2c3c(ccc2c1I)Oc1ccc2c(I)c(O)ccc2c1C3c1ccc(-c2ccccc2)cc1 ; Oc1ccc2c3c(ccc2c1)Oc1c(I)cc(I)cc1C3c1c(O)ccc2ccc(O)cc12 ; Oc1ccc2ccc3c(c2c1)Cc1cc(I)cc(I)c1O3]

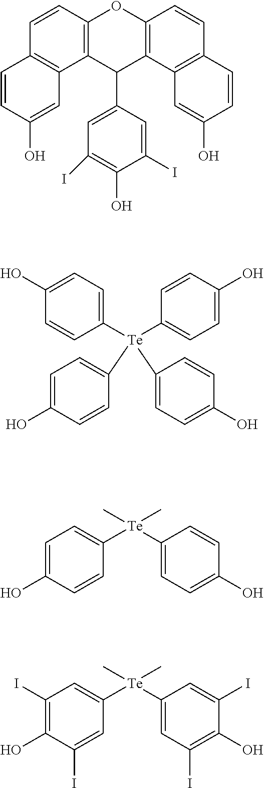

[Composition Containing Compound (A) or/and Resin (A)]

The composition of the present embodiment contains the compound (A) or/and the resin (A).

### (Solvent)

As a solvent according to the present embodiment, a publicly known solvent can be arbitrarily used as long as it can at least dissolve the compound (A) or/and the resin (A) mentioned above. Specific examples of the solvent can include, but not particularly limited to, an ethylene glycol monoalkyl ether acetate such as ethylene glycol monomethyl ether acetate, ethylene glycol monoethyl ether acetate, ethylene glycol mono-n-propyl ether acetate, and ethylene glycol mono-n-butyl ether acetate; an ethylene glycol monoalkyl ether such as ethylene glycol monomethyl ether and ethylene glycol monoethyl ether; a propylene glycol monoalkyl ether acetate such as propylene glycol monomethyl ether acetate (PGMEA), propylene glycol monoethyl ether acetate, propylene glycol mono-n-propyl ether acetate, and propylene glycol mono-n-butyl ether acetate; a propylene glycol monoalkyl ethers such as propylene glycol monomethyl ether (PGME) and propylene glycol monoethyl ether; a lactate ester such as methyl lactate, ethyl lactate, n-propyl lactate, n-butyl lactate, and n-amyl lactate; an aliphatic carboxylic acid ester such as methyl acetate, ethyl acetate, n-propyl acetate, n-butyl acetate, n-amyl acetate, n-hexyl acetate, methyl propionate, and ethyl propionate; another ester such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutyl acetate, 3-methyl-3-methoxybutyl acetate, butyl 3-methoxy-3-methylpropionate, butyl 3-methoxy methylbutyrate, methyl acetoacetate, methyl pyruvate, and ethyl pyruvate; an aromatic hydrocarbon such as toluene and xylene; a ketone such as acetone, 2-butanone, 2-heptanone, 3-heptanone, 4-heptanone, cyclopentanone (CPN), and cyclohexanone (CHN); an amide such as N,N-dimethylformamide, N-methylacetamide, N,N-dimethylacetamide, and N-methylpyrrolidone; and a lactone such as γ-lactone. The solvent used in the present embodiment is preferably a safe solvent, more preferably at least one selected from PGMEA, PGME, CHN, CPN, 2-heptanone, anisole, butyl acetate and ethyl lactate, and still more preferably at least one selected from PGMEA, PGME, CHN, CPN and ethyl lactate.

In the present embodiment, the amount of the solid component and the amount of the solvent are not particularly limited, but preferably the solid component is 1 to 80% by mass and the solvent is 20 to 99% by mass, more preferably the solid component is 1 to 50% by mass and the solvent is 50 to 99% by mass, still more preferably the solid component is 2 to 40% by mass and the solvent is 60 to 98% by mass, and particularly preferably the solid component is 2 to 10% by mass and the solvent is 90 to 98% by mass, based on the total mass of the amount of the solid component and the solvent.

### (Acid Generating Agent)

The composition of the present embodiment preferably contains one or more acid generating agents generating an acid directly or indirectly by irradiation of any radiation selected from visible light, ultraviolet, excimer laser, electron beam, extreme ultraviolet (EUV), X-ray and ion beam, or by heating. The acid generating agent is not particularly limited, and either a nonionic acid generating agent or an ionic generating agent may be used. Examples of the acid generating agent include sulfonate esters (for example, 2-nitrobenzyl ester, aromatic sulfonate, oxime sulfonate, N-sulfonyloxyimide, sulfonyloxyketone, diazonaphthoquinone 4-sulfonate), and sulfones (for example, disulfone, ketosulfone, sulfonyldiazomethane). Typical examples of the ionic acid generating agent include onium salts containing an onium cation (for example, diazonium salts, phosphonium salts, sulfonium salts, and iodonium salts). Examples of the anion of onium salts include a sulfonic acid anion, a sulfonylimide anion, and a sulfonylmethide anion. For example, the acid generating agents disclosed in International Publication No. WO 2013/024778, Japanese Patent Laid-Open No. 2009-134088, Japanese Patent Laid-Open No. 63-26653, Japanese Patent Laid-Open No. 55-164824, Japanese Patent Laid-Open No. 62-69263, Japanese Patent Laid-Open No. 63-146038, Japanese Patent Laid-Open No. 63-163452, Japanese Patent Laid-Open No. 62-153853, Japanese Patent Laid-Open No. 63-146029, U.S. Pat. Nos. 3,779,778, 3,849,137, German Patent No. 3914407, and European Patent 126,712 and the like can be used. The acid generating agents can be used alone or in combination of two or more kinds.

The amount of the acid generating agent used is preferably 0.001 to 49% by mass of the total mass of the solid component, more preferably 1 to 40% by mass, still more preferably 3 to 30% by mass, and particularly preferably 10 to 25% by mass. By using the acid generating agent within the above range, there is a tendency that curability is improved. In the present embodiment, the acid generation method is not particularly limited as long as an acid is generated in the system.

### (Acid Diffusion Controlling Agent)

Other compounds that may be used in combination may contain compounds capable of accelerating or suppressing the diffusion of generated acids as an acid diffusion controlling agent.

### <Acid Diffusion Accelerator>

Preferred acid diffusion accelerators may include a compound having a pKa value as low as 2.0 or less for the purpose of releasing the acid under necessary conditions while retaining the generated acid, having a molecular weight of 1000 or less, having a c log P value of 30 or less, or having a Tg of 250° C. or less to accelerate thermal diffusion in the resin matrix, and having heat resistance at least 250° C. As a specific structure of the acid diffusion accelerator, either a nonionic structure or an ionic structure may be used. Examples of the acid diffusion accelerator include sulfonate esters (for example, 2-nitrobenzyl ester, aromatic sulfonate, oxime sulfonate, N-sulfonyloxyimide, sulfonyloxyketone), and sulfones (for example, disulfone, ketosulfone, sulfonyldiazomethane). Typical examples of the ionic acid diffusion accelerator include onium salts containing an onium cation (for example, diazonium salts, phosphonium salts, sulfonium salts, and iodonium salts). Examples of the anion of onium salts include a sulfonic acid anion, a sulfonylimide anion, and a sulfonylmethide anion.

The cation in the ionic compound is not particularly limited as long as it satisfies any one of the above-described molecular weight, c log P, and Tg, and heat resistance in a state of forming a salt with an anion. As specific examples of the cation, an organic ammonium cation, an organic iodonium cation, and an organic sulfonium cation can be preferably used.

### <Acid Diffusion Inhibitor>

The acid diffusion inhibitor can be used for the purpose of inhibiting the diffusion of an acid generated from an acid generating agent upon exposure into a resist film or an underlayer film in each step of exposure, PEB and development, and inhibiting the reaction of a resist resin or an underlayer film resin due to the influence of an acid generated or diffused in a trace amount in an unexposed portion. Further, as other effects, it is possible to provide a resist resin composition or an underlayer film resin composition having excellent process stability, which leads to the improvement of time stability of the resist resin composition or the underlayer film resin composition, the improvement of resolution in lithography, and the improvement of process robustness by suppressing the time dependence of pattern quality required from exposure to development. The acid diffusion inhibitor may be a low molecular weight compound or may be incorporated as a part of a polymer, or both of these forms may be used in combination.

Further, as the acid diffusion inhibitor, a salt that forms an acid having weak acid dissociation properties compared to the acid generated from the acid generating agent used can be used. The acidity as an index of acid dissociation is indicated by an acid dissociation constant (pKa). In a case where a salt generating an acid weaker in acidity than the acid generated from the acid generating agent is used as the acid diffusion inhibitor, the acid dissociation constant of the acid generated from the acid diffusion inhibitor is −3<pKa, preferably −1<pKa<7, and more preferably 0<pKa<5.

Examples of the acid diffusion inhibitor include a nitrogen atom-containing compound and a photobase generating agent that generates a weak acid upon exposure to light.

Examples of the nitrogen atom-containing compound include amine compounds such as tripentylamine and trioctylamine, amide group-containing compounds such as formamide and N,N-dimethylacetamide, urea compounds such as urea and 1,1-dimethylurea, and nitrogen-containing heterocyclic compounds such as pyridine, N-(undecylcarbonyloxyethyl) morpholine and N-t-pentyloxycarbonyl-4-hydroxypiperidine.

Examples of the photobase generating agent include a compound containing an onium cation which is decomposed upon exposure and an anion of a weak acid. The photo-disintegrating base generates a weak acid from a proton generated by the decomposition of the onium cation and an anion of the weak acid in the exposed portion, and the acid diffusion controllability is thus lowered.

Examples of the salt generating an acid weaker in acidity than the acid generated from the acid generating agent include salts represented by formula (D) disclosed in Japanese Patent Laid-Open No. 2015-147926, salts disclosed in Japanese Patent Laid-Open No. 2012-229206, Japanese Patent Laid-Open No. 2012-6908, Japanese Patent Laid-Open No. 2012-72109, Japanese Patent Laid-Open No. 2011-39502, and Japanese Patent Laid-Open No. 2011-191745.

Examples of other preferred acid diffusion inhibitors include, but are not limited to, the followings:

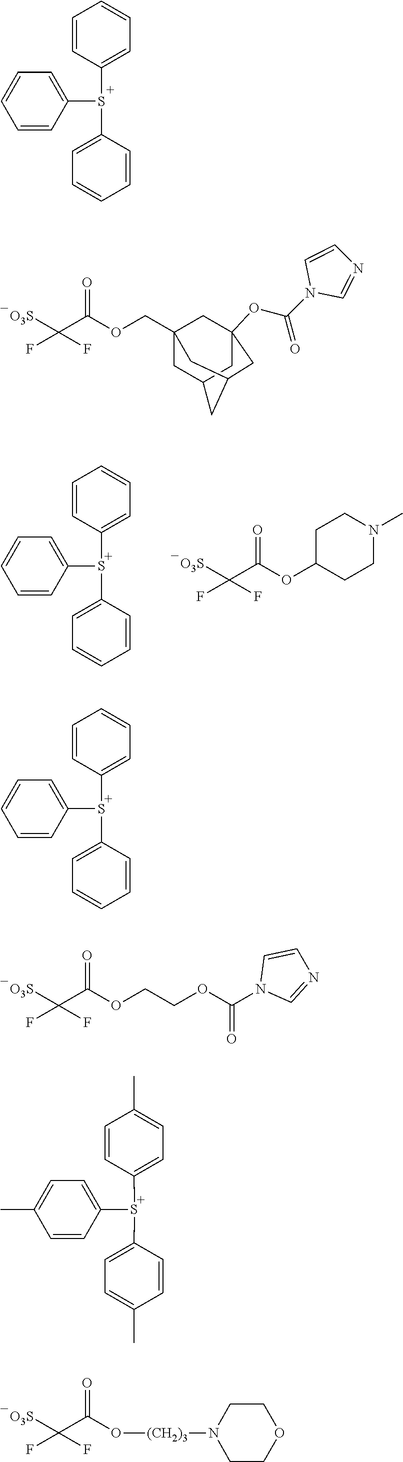

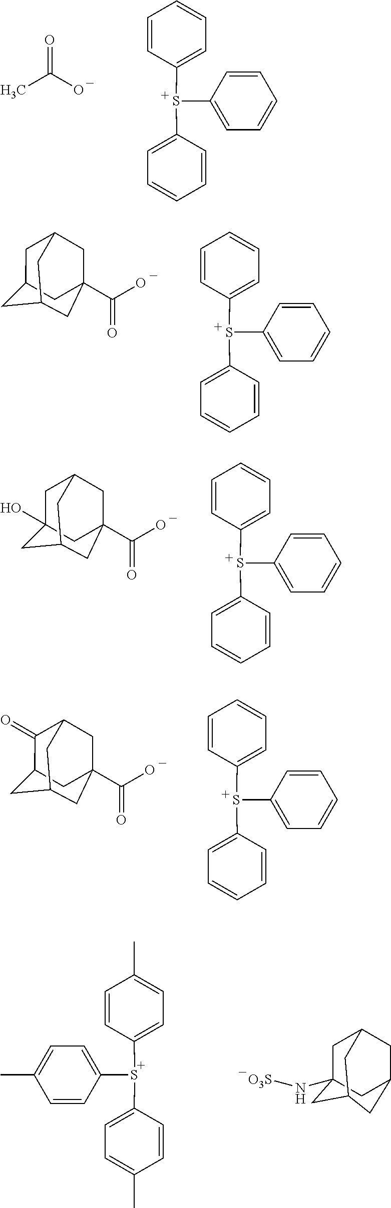

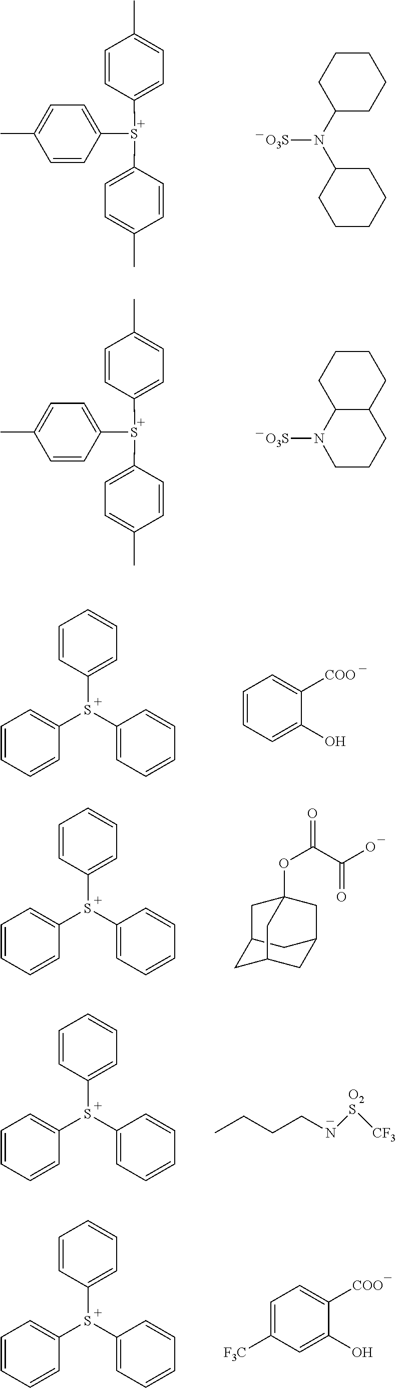

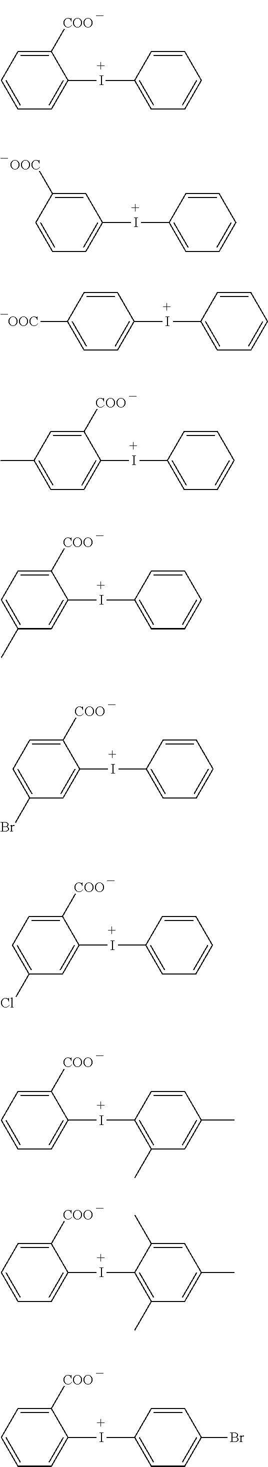

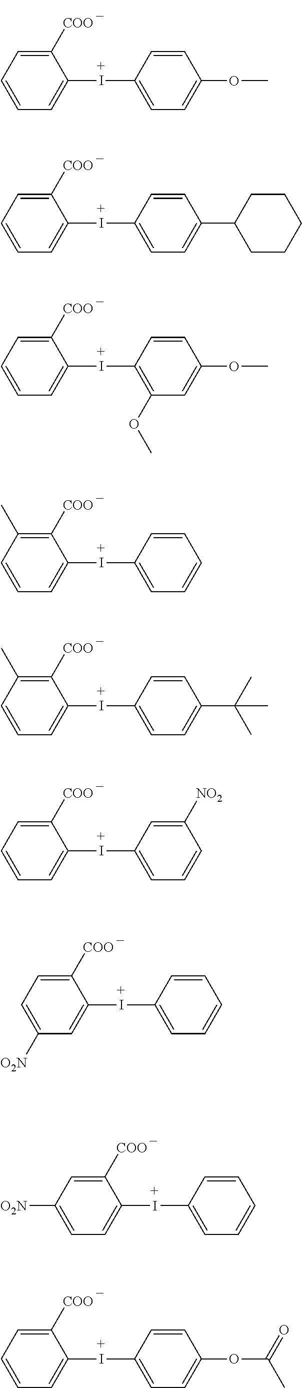

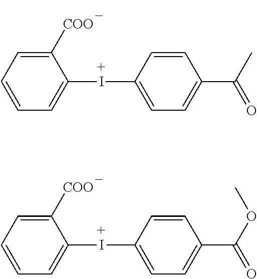

In a case where the underlayer film resin composition contains an acid diffusion controlling agent, the lower limit of the content of the acid diffusion controlling agent is preferably 0.1 parts by mass, more preferably 0.5 parts by mass, and still more preferably 1 part by mass, based on 100 parts by mass of the polymer component (or resin component). The upper limit of the content is preferably 20 parts by mass, more preferably 10 parts by mass, and more preferably 5 parts by mass.

In a case where the underlayer film resin composition contains an acid diffusion controlling agent, the lower limit of the content of the acid diffusion controlling agent is preferably 1 mol %, more preferably 5 mol %, and still more preferably 10 mol %, based on 100 mol % of the acid generating agent. The upper limit of the content is preferably 250 mol %, more preferably 150 mol %, and still more preferably 100 mol %.

When the content of the acid diffusion controlling agent is within the above range, the defect inhibition property and LWR performance of the underlayer film resin composition can be further improved. The acid diffusion controlling agent may be used alone or in combination of two or more.

### (Crosslinking Agent)

In the present embodiment, one or more crosslinking agents can be contained in the composition. The crosslinking agent refers to a compound capable of crosslinking at least either the compound (A) or the resin (A). It is preferable that the above crosslinking agent be an acid crosslinking agent capable of intramolecularly or intermolecularly crosslinking the compound (A) or the resin (A) in the presence of the acid generated from the acid generating agent. Examples of such an acid crosslinking agent can include a compound having one or more groups capable of crosslinking the compound (A) or the resin (A) (hereinafter, referred to as a “crosslinkable group”).

Examples of the above crosslinkable group can include: (i) a hydroxyalkyl group or a group derived therefrom, such as a hydroxy (alkyl group having 1 to 6 carbon atoms), alkoxy having 1 to 6 carbon atoms (alkyl group having 1 to 6 carbon atoms) and acetoxy (alkyl group having 1 to 6 carbon atoms); (ii) a carbonyl group or a group derived therefrom, such as a formyl group and a carboxy (alkyl group having 1 to 6 carbon atoms); (iii) a nitrogenous group containing group such as a dimethylaminomethyl group, a diethylaminomethyl group, a dimethylolaminomethyl group, a diethylolaminomethyl group and a morpholinomethyl group; (iv) a glycidyl group containing group such as a glycidyl ether group, a glycidyl ester group and a glycidylamino group; (v) a group derived from an aromatic group such as an allyloxy having 1 to 6 carbon atoms (alkyl group having 1 to 6 carbon atoms) and an aralkyloxy having 1 to 6 carbon atoms (alkyl group having 1 to 6 carbon atoms) such as a benzyloxymethyl group and a benzoyloxymethyl group; and (vi) a polymerizable multiple bond containing group such as a vinyl group and an isopropenyl group. As the crosslinkable group of the crosslinking agent according to the present embodiment, a hydroxyalkyl group, an alkoxyalkyl group and the like are preferable, and an alkoxymethyl group is particularly preferable.

The crosslinking agent having the above crosslinkable group is not particularly limited, and, for example, an acid crosslinking agent described in International Publication No. WO 2013/024778 can be used. The crosslinking agent can be used alone or in combination of two or more kinds.

In the present embodiment, the amount of the crosslinking agent used is preferably 0.5 to 50% by mass of the total mass of the solid component, more preferably 0.5 to 40% by mass, still more preferably 1 to 30% by mass, and particularly preferably 2 to 20% by mass. When the blending ratio of the crosslinking agent is 0.5% by mass or more, solvent resistance tends to be improved and dissolution in a resist solvent to be applied after curing can be suppressed, but when the blending ratio is 50% by mass or less, reduction in heat resistance after curing tends to be suppressed.

### (Further Component)

To the composition of the present embodiment, if required, as the further component, one or more of various additive agents such as a dissolution promoting agent, a dissolution controlling agent, a sensitizing agent, a surfactant, and an organic carboxylic acid or an oxo acid of phosphor or derivative thereof can be added.

### (Dissolution Promoting Agent)

The dissolution promoting agent is a component having a function of, when the solubility of a solid component is too low, increasing the solubility of the solid component in a developing solution to moderately increase the dissolution rate of that compound upon developing. As the above dissolution promoting agent, those having a low molecular weight are preferable, and examples thereof can include a phenolic compound having a low molecular weight. Examples of the phenolic compound having a low molecular weight can include a bisphenol and a tris(hydroxyphenyl)methane. These dissolution promoting agents can be used alone or in mixture of two or more kinds.

The content of the dissolution promoting agent, which is arbitrarily adjusted according to the kind of the above solid component to be used, is preferably 0 to 49% by mass of the total mass of the solid component, more preferably 0 to 5% by mass, still more preferably 0 to 1% by mass, and particularly preferably 0% by mass.

### (Dissolution Controlling Agent)

The dissolution controlling agent is a component having a function of, when the solubility of a solid component is too high, controlling the solubility of the solid component in a developing solution to moderately decrease the dissolution rate upon developing. As such a dissolution controlling agent, the one which does not chemically change in steps such as calcination of film, radiation irradiation, and coating of the upper layer is preferable.

The dissolution controlling agent is not particularly limited, and examples thereof can include an aromatic hydrocarbon such as phenanthrene, anthracene and acenaphthene; a ketone such as acetophenone, benzophenone and phenyl naphthyl ketone; and a sulfone such as methyl phenyl sulfone, diphenyl sulfone and dinaphthyl sulfone. These dissolution controlling agents can be used alone or in combination of two or more kinds.

The content of the dissolution controlling agent, which is arbitrarily adjusted according to the kind of the above compound to be used, is preferably 0 to 49% by mass of the total mass of the solid component, more preferably 0 to 5% by mass, still more preferably 0 to 1% by mass, and particularly preferably 0% by mass.

### (Sensitizing Agent)

The sensitizing agent is a component having a function of absorbing irradiated radiation energy, transmitting the energy to the acid generating agent, and thereby increasing the acid production amount, and improving the curability. Examples of such a sensitizing agent can include, but not particularly limited to, a benzophenone, a biacetyl, a pyrene, a phenothiazine and a fluorene. These sensitizing agents can be used alone or in combination of two or more kinds.

The content of the sensitizing agent, which is arbitrarily adjusted according to the kind of the above compound to be used, is preferably 0 to 49% by mass of the total mass of the solid component, more preferably 0 to 5% by mass, still more preferably 0 to 1% by mass, and particularly preferably 0% by mass.

### (Surfactant)

The surfactant is a component having a function of improving coatability and striation of the composition of the present embodiment, and coatability of the upper layer or the like. The surfactant may be any of anionic, cationic, nonionic, and amphoteric surfactants. Preferable examples of the surfactant include a nonionic surfactant. The nonionic surfactant has a good affinity with a solvent to be used in production of the composition of the present embodiment, and can further enhance the effects of the composition of the present embodiment. Examples of the nonionic surfactant include, but not particularly limited to, a polyoxyethylene higher alkyl ether, a polyoxyethylene higher alkyl phenyl ether, and a higher fatty acid diester of polyethylene glycol. Examples of commercially available products of these surfactants can include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.).

The content of the surfactant, which is arbitrarily adjusted according to the kind of the above solid component to be used, is preferably 0 to 49% by mass of the total mass of the solid component, more preferably 0 to 5% by mass, still more preferably 0 to 1% by mass, and particularly preferably 0% by mass.

### (Further Additive Agent)

Furthermore, the composition of the present embodiment can contain one or more of additive agents other than the components mentioned above, if required. Examples of such an additive agent include a dye, a pigment and an adhesion aid. For example, when the composition contains a dye or a pigment, a latent image of the exposed portion is visualized and influence of halation upon exposure can be alleviated, which is preferable. In addition, when the composition contains the adhesion aid, adhesiveness to a substrate and the layer in contact therewith can be improved, which is preferable. Furthermore, examples of the further additive agent can include a halation preventing agent, a storage stabilizing agent, a defoaming agent and a shape improving agent. Specific examples thereof can include 4-hydroxy-4′-methylchalkone.

In the composition of the present embodiment, the total content of the optional components can be 0 to 99% by mass of the total mass of the solid component, and is preferably 0 to 49% by mass, more preferably 0 to 10% by mass, still more preferably 0 to 5% by mass, further preferably 0 to 1% by mass, and particularly preferably 0% by mass.

The composition of the present embodiment is normally prepared by dissolving each component in a solvent upon use into a homogeneous solution, and then if required, filtering through a filter or the like with a pore diameter of about 0.2 μm, for example.

The composition of the present embodiment is used in lithography applications. The composition is preferably cured after formation of a thin film, to thereby form an underlayer film or a film in contact with a resist layer (resist layer contact film).

### (Physical Properties and the Like of Composition)

The composition of the present embodiment can form an amorphous film by spin coating. Also, the composition of the present embodiment can be applied to a general semiconductor production process. After curing, the composition of the present embodiment generates protons by irradiation with extreme ultraviolet rays and supplies protons to an adjacent layer, thereby improving the sensitivity of the adjacent layer. The composition of the present embodiment is preferably used after being cured. After curing, the composition preferably has solvent resistance so as not to dissolve in the composition of the adjacent layer.

### [Method for Producing Amorphous Film]

The composition of the present embodiment can be used to form an amorphous film on a substrate.

### [Resist Pattern Formation Method Using Composition]

The resist pattern formation method using the composition of the present embodiment comprises: an underlayer film formation step of forming an underlayer film on a substrate using the composition; a photoresist film formation step of forming at least one photoresist film on the underlayer film formed through the underlayer film formation step; and a step of irradiating a predetermined region of the photoresist film formed through the photoresist film formation step with radiation for development.

### [Circuit Pattern Formation Method Using Composition]

The circuit pattern formation method using the composition of the present embodiment comprises: an underlayer film formation step of forming an underlayer film on a substrate; a resist layer contact film formation step of forming, by using the composition, a resist layer contact film on the underlayer film formed through the underlayer film formation step; a photoresist film formation step of forming at least one photoresist film on the resist layer contact film formed through the resist layer contact film formation step; a resist pattern formation step of irradiating a predetermined region of the photoresist film formed through the photoresist film formation step with radiation for development, thereby forming a resist pattern; a pattern formation step of etching the resist layer contact film or the resist layer contact film and the underlayer film with the resist pattern formed through the resist pattern formation step as a mask, thereby forming a pattern; and a substrate pattern formation step of etching the substrate with the pattern formed through the pattern formation step as a mask, thereby forming a pattern on the substrate.

### EXAMPLES

The present embodiment will be described in more detail with reference to synthesis examples and examples below. However, the present embodiment is not limited to these examples by any means.

### [Measurement Method]

### (1) Structure of Compound

The structure of a compound was confirmed by 1H-NMR measurement using Advance III 500 manufactured by Bruker Corp. under the following conditions.

Frequency: 500 MHz

Solvent: d6-DMSO

Internal standard: TMS

Measurement temperature: 23° C.

### [Evaluation Method]

### (Preparation of Underlayer Film Composition)

Ten parts by mass of the compound or polymer obtained in the following Synthesis Working Examples, 0.2 parts by mass of a thermal acid generator TAG-2689 (manufactured by King Industries, Inc., quaternary ammonium trifluoromethanesulfonate), 1 part by mass of TMOM-BP (manufactured by Honshu Chemical Industry Co., Ltd.), 76.8 parts by mass of PGMEA, and 12 parts by mass of PGME were mixed to prepare an underlayer film composition solution containing the compound of the present invention.

For the compound of Example 4, WPBG300 (0.2 parts by mass) was added instead of TAG-2689, and BPNO1S (1 part by mass) was added instead of TMOM-BP.

### (1) Safe Solvent Solubility Test of Compound

The solubility of the compound in PGMEA was evaluated according to the following criteria using the amount dissolved in each solvent. The amount of dissolution was measured at 23° C. by precisely weighing the compound into a test tube, adding a target solvent so as to attain a predetermined concentration, applying ultrasonic waves for 30 minutes in an ultrasonic cleaner, and then visually observing the subsequent state of the fluid.

A: 5.0% by mass≤Amount of dissolution

B: 2.0% by mass≤Amount of dissolution<5.0% by mass

C: Amount of dissolution<2.0% by mass

### (2) Storage Stability and Thin Film Formability of Composition

The storage stability of the composition containing the compound or resin was evaluated by leaving the composition after preparation of the above underlayer film composition to stand still at 23° C. for 3 days, and visually observing the presence or absence of precipitates. A clean silicon wafer was spin coated with the composition, and then baked on a hot plate at 250° C. to form an amorphous film having a thickness of 100 nm. The prepared composition was evaluated as ◯ in a case where it was a uniform solution and thin film formability was good, Δ when it was a uniform solution but there was a defect in the thin film, and X when there was precipitation.

### (3) Sensitivity Evaluation

### (EUV Sensitivity—TMAH Aqueous Solution Development)

A resist solution for sensitivity evaluation and pattern evaluation was prepared by blending 5 parts by mass of the polymer MAR1 obtained in the following Synthesis Working Example of polymer for resists, 1 part by mass of triphenylsulfonium nonafluoromethanesulfonate, 0.2 part by mass of tributylamine, 80 parts by mass of PGMEA, and 12 parts by mass of PGME.

The underlayer film composition was applied onto a silicon wafer and baked at 240° C. for 60 seconds to form an underlayer film having a thickness of 100 nm on the silicon wafer.

Furthermore, this underlayer film of the present invention formed on a silicon wafer was coated with a resist solution and baked at 110° C. for 60 seconds to form a photoresist layer with a film thickness of 100 nm.

Then, after performing a maskless shot exposure with an increased exposure amount from 1 mJ/cm² to 80 mJ/cm² by 1 mJ/cm² at a time using an extreme ultraviolet (EUV) exposure apparatus “EUVES-7000” (product name, manufactured by Lithotech Japan Co., Ltd.), the wafer was baked at 110° C. for 90 seconds (PEB), developed with 2.38% by mass tetramethylammonium hydroxide (TMAH) aqueous solution for 60 seconds, and a wafer on which a shot exposure for 80 shots was performed was obtained. The film thickness of each shot exposure area thus obtained was measured by an optical interference film thickness meter “VM 3200” (product name, manufactured by SCREEN Semiconductor Solutions Co., Ltd.), profile data of the film thickness with respect to the exposure amount was obtained, and the exposure amount at which the gradient of the film thickness variation amount with respect to the exposure amount became the largest was calculated as a sensitivity value (mJ/cm²), and used as an index of the EUV sensitivity of the resist.

### (4) Etching Resistance

Etching apparatus: RIE-10NR manufactured by Samco International, Inc.

Output: 50 W

Pressure: 20 Pa

Time: 2 min

Etching gas

Ar gas flow rate:CF₄ gas flow rate:O₂ gas flow rate=50:5:5 (sccm)

A film formed on a silicon wafer using the underlayer film solution composed of the material of the present invention prepared in each of Examples and Comparative Examples was subjected to an etching test under the above conditions, and the etching rate at that time was measured. Then, the etching resistance was evaluated according to the following evaluation criteria on the basis of the etching rate of an underlayer film prepared by using a novolac (“PSM4357” manufactured by Gunei Chemical Industry Co., Ltd.).

### Evaluation Criteria

A: The difference in etching rate is smaller than that of the underlayer film of novolac by 10% or more in terms of the ratio to novolac.

B: The difference in etching rate is within ±10% as compared with that of the underlayer film of novolac in terms of the ratio to novolac.

C: The difference in etching rate is larger than 10% as compared with that of the underlayer film of novolac in terms of the ratio to novolac.

### Synthesis Working Example 1: Synthesis of BisB-1

In a container (internal capacity: 200 mL) equipped with a stirrer, a condenser tube, and a burette, 25.0 g (204.7 mmol) of 2,6-dimethylphenol (a reagent manufactured by Tokyo Chemical Industry Co., Ltd.), 25.0 g (107.7 mmol) of 4-iodobenzaldehyde (a reagent manufactured by Tokyo Chemical Industry Co., Ltd.), and 20 mL of 1-methoxy-2-propanol were placed, and 5.3 g (53.9 mmol) of sulfuric acid was added to prepare a reaction liquid. This reaction liquid was stirred at 90° C. for 6 hours and reacted. After the reaction finished, 1 L of pure water was added to the reaction liquid, and sodium bicarbonate was added under ice cooling such that the pH was adjusted to 7 to 8. Extraction with ethyl acetate was performed, followed by concentration, to obtain a solution. The obtained solution was separated and purified by column chromatography to obtain 24.9 g of the objective compound (BisB-1) represented by the following formula. The following peaks were found by NMR measurement performed on the obtained compound (BisB-1) under the above measurement conditions, and the compound was confirmed to have a chemical structure of the following formula (BisB-1).

δ (ppm) 8.1 (2H, —O—H), 6.5-7.7 (8H, Ph-H), 5.2 (1H, C—H), 2.1 (12H, CH3)

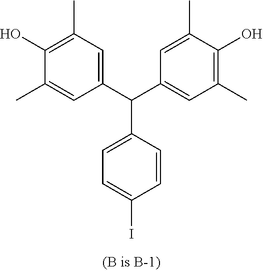 [SMILES: Cc1cc(C(c2ccc(I)cc2)c2cc(C)c(O)c(C)c2)cc(C)c1O]

### Synthesis Working Example 2: Synthesis of BisB-2

In a container (internal capacity: 500 mL) equipped with a stirrer, a condenser tube, and a burette, 42.8 g (230 mmol) of 4,4′-biphenol (a reagent manufactured by Tokyo Chemical Industry Co., Ltd.), 21.5 g (57.5 mmol) of 3,5-diiodosalicylaldehyde (a reagent manufactured by Tokyo Chemical Industry Co., Ltd.), and 428 mL of γ-butyrolactone were placed, and 5.8 g (58 mmol) of sulfuric acid was added to prepare a reaction liquid. This reaction liquid was stirred at 90° C. for 56 hours and reacted. After the reaction finished, 1 L of pure water was added to the reaction liquid, which was then neutralized with sodium hydroxide. Extraction with ethyl acetate was performed, followed by concentration, to obtain a solution. The obtained solution was separated and purified by column chromatography to obtain 10 g of the objective compound (BisB-2) represented by the following formula. The following peaks were found by NMR measurement performed on the obtained compound (BisB-2) under the above measurement conditions, and the compound was confirmed to have a chemical structure of the following formula (BisB-2).

δ (ppm) 9.4 (4H, —O—H), 8.9 (1H, —O—H), 6.2-7.8 (16H, Ph-H), 6.3 (1H, C—H)

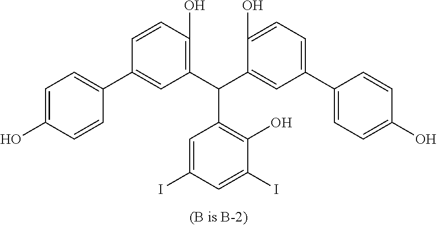 [SMILES: Oc1ccc(-c2ccc(O)c(C(c3cc(-c4ccc(O)cc4)ccc3O)c3cc(I)cc(I)c3O)c2)cc1]

### Synthesis Working Example 3: Synthesis of XbisN-1

To a container (internal capacity: 300 ml) equipped with a stirrer, a condenser tube, and a burette, 7.0 g (40 mmol) of 2,6-naphthalenediol (a reagent manufactured by Sigma-Aldrich) and 4.6 g (20 mmol) of 3-iodobenzaldehyde (a reagent manufactured by Tokyo Chemical Industry Co., Ltd.) were charged in 100 ml of γ-butyrolactone, and 0.5 g of p-toluenesulfonic acid was added thereinto, and the reaction was carried out with stirring at 90° C. for 23 hours to obtain a reaction solution. The reaction solution was then added to 1000 g of pure water, extracted with ethyl acetate, and concentrated to obtain a solution.

The obtained solution was separated by column chromatography and then washed with chloroform to obtain 4.2 g of the objective compound (XbisN-1) represented by the following formula (XbisN-1). As a result of measuring the molecular weight of the obtained compound (XbisN-1) by the above method, the molecular weight was 516.

The following peaks were found by NMR measurement performed on the obtained compound (XbisN-1) under the above measurement conditions, and the compound was confirmed to have a chemical structure of the following formula (XbisN-1).

δ (ppm) 9.7 (2H, O—H), 7.0-8.5 (14H, Ph-H), 6.5 (1H, C—H)

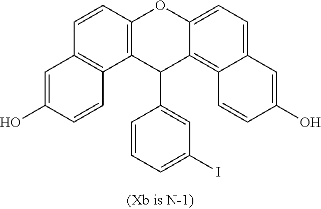 [SMILES: Oc1ccc2c3c(ccc2c1)Oc1ccc2cc(O)ccc2c1C3c1cccc(I)c1]

### Synthesis Working Example 4: Synthesis of BMI-1

A 200 mL glass flask was used as a reaction container, and 5.73 g (20 mmol) of neopentyl glycol bis (4-aminophenyl) ether (product name: DANPG, manufactured by SEIKA CORPORATION) was dissolved in butanol as a solvent, and 20% by mass iodine aqueous chloride solution (81.2 g, 100 mmol) was added dropwise at 50° C. over a period of 60 minutes, followed by stirring at 50° C. for 2 hours to react salicyl alcohol with iodine chloride. An aqueous solution of sodium thiosulfate was added to the reaction solution after the reaction, and the mixture was stirred for 1 hour and then cooled to 10° C. The precipitate precipitated by cooling was filtered off, washed, and dried to obtain 9.5 g of a brown solid. As a result of analyzing samples of the brown solid by liquid chromatography-mass spectrometry (LC-MS), a compound (X) represented by the following formula (X) was confirmed.

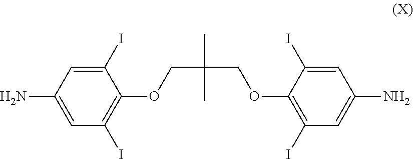 [SMILES: CC(C)(COc1c(I)cc(N)cc1I)COc1c(I)cc(N)cc1I]

Next, the compound (X) obtained above was transferred to a container (internal capacity: 200 ml) equipped with a stirrer, a condenser tube, and a burette, 2.54 g (26.0 mmol) of maleic anhydride (manufactured by KANTO CHEMICAL CO., INC.), 50 ml of dimethylformamide and 50 ml of m-xylene were added thereto, and 0.5 g (2.9 mmol) of p-toluenesulfonic acid was added to prepare a reaction solution. The reaction solution was stirred at 130° C. for 4.0 hours to conduct reaction, and the produced water was recovered with a Dean-and-stark trap through azeotropic dehydration. Next, the reaction solution was cooled to 40° C., and it was then added dropwise into a beaker in which 500 ml of distilled water was placed to precipitate the product. After filtering the obtained slurry solution, the residue was washed with methanol and subjected to separation and purification with column chromatography to acquire 1.5 g of the objective compound (BMI-1) represented by the following formula:

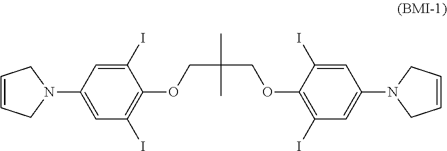 [SMILES: CC(C)(COc1c(I)cc(N2CC=CC2)cc1I)COc1c(I)cc(N2CC=CC2)cc1I]

The following peaks were found by NMR measurement performed on the obtained compound (BMI-1) under the above measurement conditions, and the compound was confirmed to have a chemical structure of the following formula (BMI-1).

1H-NMR: (d-DMSO, Internal standard TMS)

1H-NMR: (d-DMSO, Internal standard TMS)

δ (ppm) 7.0-7.5 (4H, Ph-H), 3.2 (4H, —CH═CH), 2.4 (4H, —CH2-), 1.6-1.7 (6H, CH3-C—CH3)

### Synthesis Working Example 5: Synthesis of XBisN-2

To a container (internal capacity: 500 mL) equipped with a stirrer, a condenser tube, and a burette, 32.0 g (20 mmol) of 2,6-naphthalenediol (a reagent manufactured by Sigma-Aldrich), 29.9 g (80 mmol) of 3,5-diiodosalicylaldehyde (manufactured by Tokyo Chemical Industry Co., Ltd.), and 200 mL of 1,4-dioxane were charged, and 10 mL of 95% sulfuric acid was added, and the reaction was carried out with stirring at 100° C. for 6 hours. Next, the reaction solution was neutralized with a 24% aqueous sodium hydroxide solution, 100 g of pure water was added to precipitate the reaction product, and after cooling to room temperature, the precipitates was separated by filtration. The obtained solid matter was dried and then separated and purified by column chromatography to obtain 2.6 g of the objective compound (XBisN-2) represented by the following formula.

The following peaks were found by 500 MHz-1H-NMR, and the compound was confirmed to have a chemical structure of the following formula.

1H-NMR: (d-DMSO, Internal standard TMS)

δ (ppm) 9.6-9.7 (3H, O—H), 6.7-8.5 (12H, Ph-H), 6.2 (1H, C—H)

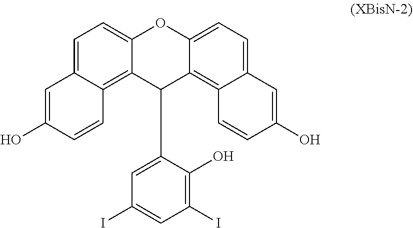 [SMILES: Oc1ccc2c3c(ccc2c1)Oc1ccc2cc(O)ccc2c1C3Cc1cc(I)cc(I)c1O]

### Synthesis Working Example 5-1: Synthesis of RXBisN-2

To a container (internal capacity: 100 mL) equipped with a stirrer, a condenser tube, and a burette, 2.6 g (7.0 mmol) of XBisN-2 and 1.0 g (2 mmol) of monobutyl copper phthalate were charged, and 20 mL of 1 butanol was added as a solvent, and the reaction solution was stirred at 100° C. for 6 hours and reacted. After cooling, the precipitate was filtered and the resulting crude was dissolved in 20 mL of ethyl acetate. Next, 1 mL of hydrochloric acid was added, and after stirring at room temperature, neutralization was performed with sodium hydrogen carbonate. The ethyl acetate solution was concentrated, 40 mL of methanol was added to precipitate the reaction product, cooled to room temperature, and separated by filtration. The obtained solid matter was dried to obtain 1.0 g of an objective resin (RXBisN-2) having a structure represented by the following formula.

As a result of measuring the molecular weight of the obtained resin in terms of polystyrene by the above method, Mn was 4300, Mw was 5500, and Mw/Mn was 1.28.

The following peaks were found by NMR measurement performed on the obtained resin under the above measurement conditions, and the compound was confirmed to have a chemical structure of the following formula.

δ (ppm) 9.5-9.7 (3H, O—H), 6.7-8.5 (12H, Ph-H), 6.0-6.3 (1H, C—H)

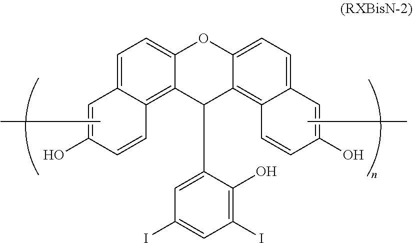

### Synthesis Working Example 6: Synthesis of NAFP-AL

In a nitrogen gas atmosphere, 1,4-bis(chloromethyl)benzene (28.8 g, 0.148 mol, manufactured by Tokyo Chemical Industry Co., Ltd.), 1-naphthol (30.0 g, 0.1368 mol, manufactured by Tokyo Chemical Industry Co., Ltd.), and p-toluenesulfonic acid monohydrate (5.7 g, 0.029 mol, manufactured by Tokyo Chemical Industry Co., Ltd.) were added to a 300 mL four-neck flask, and 150.4 g of propylene glycol monomethyl ether acetate (hereinafter abbreviated as PGMEA) was further added thereto, and the mixture was stirred, heated until reflux was confirmed, dissolved, so that polymerization was initiated. After 16 hours, the mixture was allowed to cool to 60° C., and reprecipitated into 1600 g of methanol.

The obtained precipitate was filtered and dried in a vacuum dryer at 60° C. for 16 hours to obtain 38.6 g of an oligomer having a structural unit represented by formula (NAFP-AL). The obtained oligomer had a weight average molecular weight of 2020 and a dispersity of 1.86 measured in terms of polystyrene by GPC.

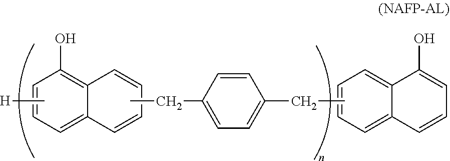

### Synthesis Working Example 6-1: Synthesis of I-NAFP-AL

A 200 mL glass flask was used as a reaction container, and 8 g (20 mmol) of NAFP-AL obtained above was dissolved in butanol as a solvent, and 20% by mass iodine aqueous chloride solution (81.2 g, 100 mmol) was added dropwise at 50° C. over a period of 60 minutes, followed by stirring at 50° C. for 2 hours to react salicyl alcohol with iodine chloride. An aqueous solution of sodium thiosulfate was added to the reaction solution after the reaction, and the mixture was stirred for 1 hour and then cooled to 10° C. The precipitate precipitated by cooling was filtered off, washed, and dried to obtain 11.5 g of a brown solid.

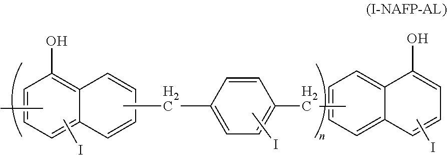

### Synthesis Working Example 7: Synthesis of 2I-PHS

A 200 mL glass flask was used as a reaction container, and 4.96 g (40 mmol) of salicyl alcohol was dissolved in butanol as a solvent, and 20% by mass iodine aqueous chloride solution (81.2 g, 100 mmol) was added dropwise at 50° C. over a period of 60 minutes, followed by stirring at 50° C. for 2 hours to react salicyl alcohol with iodine chloride. An aqueous solution of sodium thiosulfate was added to the reaction solution after the reaction, and the mixture was stirred for 1 hour and then cooled to 10° C. The precipitate precipitated by cooling was filtered off, washed, and dried to obtain 12.1 g of a white solid. As a result of analyzing samples of the white solid by liquid chromatography-mass spectrometry (LC-MS), 4-hydroxy-3,5-diiodobenzyl alcohol was confirmed.

After adding MnO2 (3.4 g, 40 mmol) in a methylene chloride solvent and stirring, a 50% by mass solution in which the whole amount of the synthesized 4-hydroxy-3,5-diiodobenzyl alcohol was dissolved in methylene chloride was added dropwise and stirred for 1 hour, and after stirring for 4 hours at room temperature, the reaction solution was filtered off and the solvent was distilled off to obtain 4-hydroxy-3,5-diiodobenzaldehyde.

A solution in which dimethyl malonate (5.3 g, 40 mmol) and the whole amount of 4-hydroxy-3,5-diiodobenzaldehyde synthesized above were dissolved was prepared in a DMF solvent, and then a solution in which ethylenediamine (0.3 g) was dissolved in DMF was stirred for 1 hour while dropping the solution, and then the solution was stirred for 6 hours while controlling the temperature of the solution to 150° C. with an oil bath and allowed to react. Thereafter, after ethyl acetate and water were added, a 2 mol/L aqueous solution of HCl was added to control the pH to be 4 or less, and the organic phase was separated by a liquid separation operation. The obtained organic phase was washed by separating 2 mol/L aqueous sodium carbonate solution, water and brine in this order, followed by filter purification and distillation of the solvent from the organic phase to obtain 8.1 g of compound 2I-PHS (4-hydroxy-3,5-diodostyrene) represented by the following formula (2I-PHS).

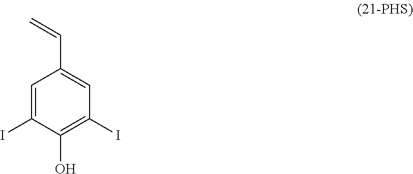

### Synthesis Working Example 7-1: Synthesis of P-2I-PHS-MMA

Three grams of compound 2I-PHS and 1.2 g of methyl methacrylate were dissolved in 45 mL of tetrahydrofuran, and azobisisobutyronitrile 0.20 g was added. After refluxing for 12 hours, the reaction solution was added dropwise to 2 L of n-heptane. The precipitated polymer was filtered off and dried under reduced pressure to obtain a white powdery polymer P-2I-PHS-MMA represented by the following formula (P-2I-PHS-MMA). The polymer had a weight average molecular weight (Mw) of 8000 and a dispersity (Mw/Mn) of 1.50. As a result of measuring 13C-NMR, the composition ratio (molar ratio) in the following formula (P-2I-PHS-MMA) was a:b=1:1. Although the following formula (P-PHS-MMA) shown below is briefly described in order to indicate the ratio of each constituent unit, the arrangement order of each constituent unit is random, and P-2I-PHS-MMA is not a block copolymer in which each constituent unit forms an independent block.

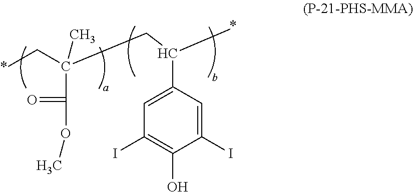

### Synthesis Working Example 8: Synthesis of XBisN-3

To a container (internal capacity: 3 L) equipped with a stirrer, a condenser tube, and a burette, 100 g (0.214 mol) of a compound (XBisN-C1) disclosed in International Publication No. WO 2013/024779 and represented by the following formula and 71.2 g (0.429 mol) of potassium iodide were charged, 1 L of methanol was added as a solvent, and 146 g (1.5 mol) of sulfate was further added dropwise under ice cooling, and the mixture was stirred at 10° C. for 4 hours and reacted. After completion of the reaction, the mixture was extracted with butyl acetate, washed with water, neutralized, and then filtered and dried to obtain 87.8 g of the objective compound (RBisN-3) and represented by the following formula (XBisN-3).

The following peaks were found by NMR measurement performed on the obtained compound under the above measurement conditions, and the compound was confirmed to have a chemical structure of the following formula.

δ (ppm) 10.6 (2H, O—H), 7.2-8.6 (17H, Ph-H), 6.7 (1H, C—H)

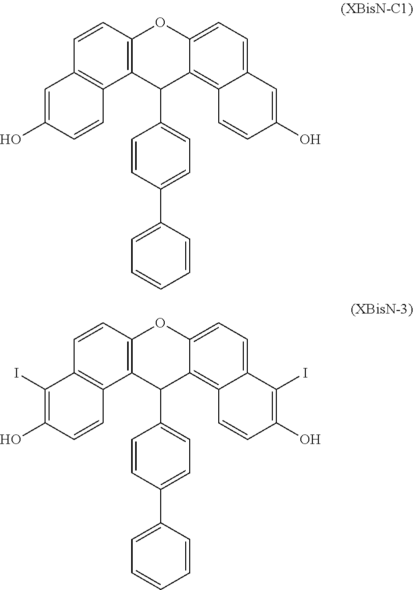

### Synthesis Working Example 9-1: Synthesis of MAC-ADI

In 200 mL of toluene, 16.8 g (0.1 mol) of 1,3-adamantanediol (manufactured by Mitsubishi Gas Chemical) was added with 89.8 g (0.4 mol) of a 57% aqueous solution of hydrogen iodide, and the mixture was stirred at 80° C. for 8 hours to react. After the reaction, water was added, washing was performed with sodium hydrogen carbonate, and the organic layer was concentrated and then separated and purified by column chromatography to obtain 12 g of 3-iodo-1-hydroxyadamantane represented by the following formula.

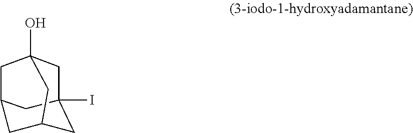 [SMILES: OC12CC3CC(C1)CC(I)(C3)C2]

2.78 g (10 mmol) of 3-iodo-1-hydroxyadamantane obtained above was dissolved in chloroform, to the mixture was added 0.96 g (12 mmol) of pyridine under ice-cooling, and 1.25 g (12 mmol) of methacrylic acid chloride was added dropwise thereto. Subsequently, the mixture was stirred and reacted under ice-cooling for 1 hour and at room temperature for 3 hours. After completion of the reaction, water was added to the reaction liquid, and the mixture was washed with saturated aqueous sodium hydrogen carbonate solution. The organic phase was dried over sodium sulfate, concentrated, and purified by column chromatography to obtain 2.7 g of the objective product (MAC-ADI) shown below.

When the obtained compound (MAC-ADI) was subjected to NMR measurement under the above measurement conditions, the following peaks were found, and it was confirmed that the compound had a chemical structure represented by formula (MAC-ADI):

δ (ppm) (d-DMSO): 6.4-6.5 (2H, ═CH2), 1.3-3.2 (17H, Ad-H, —C(CH3)═C)

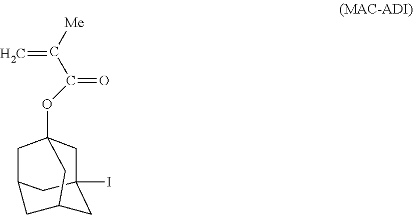 [SMILES: C=C(C)C(=O)OC12CC3CC(CC(I)(C3)C1)C2]

### Synthesis Working Example 10-1: Synthesis of MAC-ADI2

In 100 mL of toluene, 2.3 g (12.5 mmol) of 1,3,5-adamantanetriol (manufactured by Mitsubishi Gas Chemical) was added with 28.1 g (125 mmol) of a 57% aqueous solution of hydrogen iodide, and the mixture was stirred at 80° C. for 13 hours to react. After the reaction, water was added, washing was performed with sodium hydrogen carbonate, and the organic layer was concentrated and then separated and purified by column chromatography to obtain 0.9 g of 3,5-diiodo-1-hydroxyadamantane represented by the following formula.

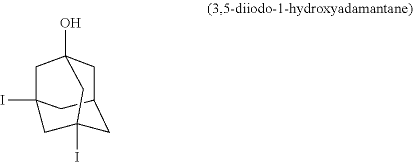 [SMILES: OC12CC3CC(I)(C1)CC(I)(C3)C2]

In the same manner as in Synthesis Example 9-1 except that 4.04 g (10 mmol) of 3,5-diiodo-1-hydroxyadamantane obtained above was used instead of 2.78 g of 3-iodo-1-hydroxyadamantane, 3.5 g of an objective compound (MAC-ADI2) represented by the following formula (MAC-ADI2) was obtained.

When the obtained compound (MAC-ADI2) was subjected to NMR measurement under the above measurement conditions, the following peaks were found, and it was confirmed that the compound had a chemical structure represented by formula (MAC-ADI2):

δ (ppm) (d-DMSO): 6.4-6.5 (2H, ═CH2), 1.5-3.9 (16H, Ad-H, —C(CH3)═C)

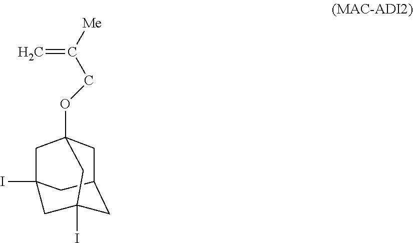 [SMILES: C=C(C)COC12CC3CC(I)(CC(I)(C3)C1)C2]

### Synthesis Working Example 9: Synthesis of P-MAC-ADI Resin

In 45 mL of tetrahydrofuran, MAC-ADI (4.2 g), 1.5 g of 2-methyl-2-adamantyl methacrylate, 2.0 g of γ-butyrolactone methacrylate and 1.5 g of hydroxyadamantyl methacrylate were dissolved, and 0.20 g of azobisisobutyronitrile was added thereto. After refluxing for 12 hours, the reaction solution was added dropwise to 2 l of n-heptane. The deposited resin was filtered off and dried under reduced pressure to obtain a white powdery resin represented by the following chemical formula (P-MAC-ADI). The resin had a molecular weight (Mw) of 9300 and a dispersity (Mw/Mn) of 1.9. As a result of measuring ¹³C-NMR, the composition ratio (molar ratio) in the following chemical formula (P-MAC-ADI) was a:b:c:d=20:30:15:35. Although the chemical formula (P-MAC-ADI) shown below is briefly described in order to indicate the ratio of each constituent unit, P-MAC-ADI is not a block copolymer in which each constituent unit forms an independent block.

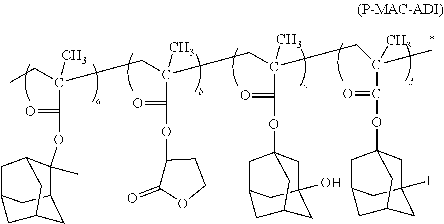

### Synthesis Working Example 10: Synthesis of P-MAC-ADI2 Resin

In 45 mL of tetrahydrofuran, MAC-ADI (5.6 g), 1.5 g of 2-methyl-2-adamantyl methacrylate, 2.0 g of γ-butyrolactone methacrylate and 1.5 g of hydroxyadamantyl methacrylate were dissolved, and 0.20 g of azobisisobutyronitrile was added thereto. After refluxing for 12 hours, the reaction solution was added dropwise to 2 l of n-heptane. The deposited resin was filtered off and dried under reduced pressure to obtain a white powdery resin represented by the following chemical formula (P-MAC-ADI2). The resin had a molecular weight (Mw) of 8350 and a dispersity (Mw/Mn) of 2.0. As a result of measuring ¹³C-NMR, the composition ratio (molar ratio) in the following chemical formula (MAC-ADI2) was a:b:c:d=20:30:15:35. Although the chemical formula (P-MAC-ADI2) shown below is briefly described in order to indicate the ratio of each constituent unit, P-MAC-ADI2 is not a block copolymer in which each constituent unit forms an independent block.

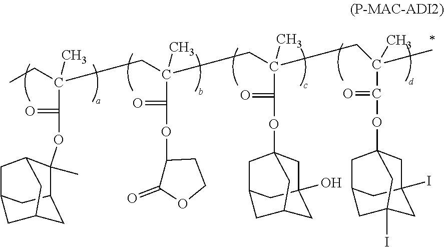

### Synthesis Comparative Example AR1: Synthesis of P-PHS-MMA

One gram of p-hydroxystyrene and 1.2 g of methyl methacrylate were dissolved in 45 mL of tetrahydrofuran, and azobisisobutyronitrile 0.20 g was added. After refluxing for 12 hours, the reaction solution was added dropwise to 2 L of n-heptane. The precipitated polymer was filtered off and dried under reduced pressure to obtain a white powdery polymer P-PHS-MMA represented by the following formula (P-PHS-MMA). The polymer had a weight average molecular weight (Mw) of 9100 and a dispersity (Mw/Mn) of 1.60. As a result of measuring 13C-NMR, the composition ratio (molar ratio) in the following formula (P-PHS-MMA) was a:b=1:1. Although the following formula (P-PHS-MMA) is briefly described in order to indicate the ratio of each constituent unit, the arrangement order of each constituent unit is random, and P-PHS-MMA is not a block copolymer in which each constituent unit forms an independent block.

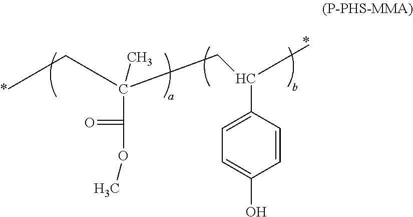

### (Synthesis of Resist Polymer MAR1)

In 45 mL of tetrahydrofuran, 0.5 g of p-hydroxystyrene (manufactured by Toho Chemical Industry Co., Ltd.), 3.0 g of 2-methyl-2-adamantyl methacrylate, 2.0 g of γ-butyrolactone methacrylate and 1.5 g of hydroxyadamantyl methacrylate were dissolved, and 0.20 g of azobisisobutyronitrile was added thereto. After refluxing for 12 hours, the reaction solution was added dropwise to 2 L of n-heptane. The precipitated polymer was filtered off and dried under reduced pressure to obtain a white powdery polymer MAR1 represented by the following formula (MAR1). The polymer had a weight average molecular weight (Mw) of 1,2000 and a dispersity (Mw/Mn) of 1.90. As a result of measuring ¹³C-NMR, the composition ratio (molar ratio) in the following formula (MAR1) was a:b:c:d=40:30:15:15. Although the following formula (MAR1) is briefly described in order to indicate the ratio of each constituent unit, the arrangement order of each constituent unit is random, and MAR1 is not a block copolymer in which each constituent unit forms an independent block. The molar ratio was determined based on the integral ratio of the carbon at the root of the benzene ring in the polystyrene-based monomer (p-hydroxystyrene) and the carbonyl carbon of the ester bond in the methacrylate-based monomers (2-methyl-2-adamantyl methacrylate, γ-butyrolactone methacrylate, and hydroxyadamantyl methacrylate).

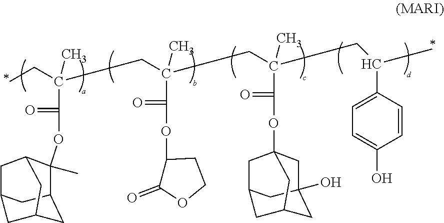

### (Synthesis of Resist Polymer MAR2)

In 45 mL of tetrahydrofuran, 1.0 g of p-hydroxystyrene (manufactured by Toho Chemical Industry Co., Ltd.), 3.8 g of 2-methyl-2-adamantyl methacrylate, 0.7 g of γ-butyrolactone methacrylate and 1.0 g of hydroxyadamantyl methacrylate were dissolved, and 0.20 g of azobisisobutyronitrile was added thereto. After refluxing for 12 hours, the reaction solution was added dropwise to 2 L of n-heptane. The precipitated polymer was filtered off and dried under reduced pressure to obtain a white powdery polymer MAR2 represented by the following formula (MAR2). The polymer had a weight average molecular weight (Mw) of 1,2000 and a dispersity (Mw/Mn) of 1.90. As a result of measuring ¹³C-NMR, the composition ratio (molar ratio) in the following formula (MAR2) was a:b:c:d=50:10:10:30. Although the following formula (MAR2) is briefly described in order to indicate the ratio of each constituent unit, the arrangement order of each constituent unit is random, and MAR2 is not a block copolymer in which each constituent unit forms an independent block. The molar ratio was determined based on the integral ratio of the carbon at the root of the benzene ring in the polystyrene-based monomer (p-hydroxystyrene) and the carbonyl carbon of the ester bond in the methacrylate-based monomers (2-methyl-2-adamantyl methacrylate, γ-butyrolactone methacrylate, and hydroxyadamantyl methacrylate).

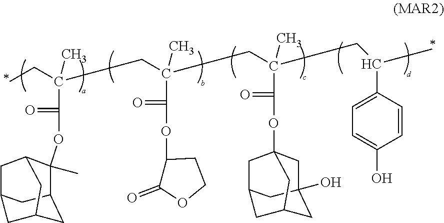

### Examples 1 to 10 and Comparative Example 1

The compounds or resins obtained in Synthesis Working Examples 1 to 10 and Synthesis Comparative Example AR1 were evaluated for safe-solvent solubility, storage stability, thin film formability, sensitivity, and etching resistances as described above. The results are shown in Table 1.

### Comparative Example 2

The evaluation was performed in the same manner as in Examples 1 to 10 except that the compound (XBisN-C1) represented by the following formula disclosed in International Publication No. WO 2013/024779 was used instead of the compounds or resins obtained in Synthesis Working Examples 1 to 10. The results are shown in Table 1.

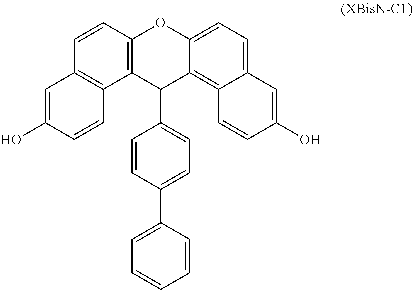 [SMILES: Oc1ccc2c3c(ccc2c1)Oc1ccc2cc(O)ccc2c1C3c1ccc(-c2ccccc2)cc1]

TABLE 1

| | | Safety | Storage | | |
|---|---|---|---|---|---|
| | | solvent | Stability/ | | |
| | Compound | solubility | Thin film | Sensitivity | Etch |
| | or Resin | PGMEA | formability | mJ/cm2 | resistance |
| Example 1 | BisB-1 | A | ◯ | 25 | B |
| Example 2 | BisB-2 | A | ◯ | 23 | B |
| Example 3 | XbisN-1 | B | ◯ | 24 | A |
| Example 4 | BMI-1 | A | ◯ | 19 | C |
| Example 5 | RXBisN-2 | A | ◯ | 22 | A |
| Example 6 | I-NAFP-AL | A | ◯ | 21 | A |
| Example 7 | P-2I-PHS- | A | ◯ | 23 | C |
| | MMA | | | | |
| Example 8 | XBisN-3 | A | ◯ | 23 | A |
| Example 9 | P-MAC- | A | ◯ | 23 | C |
| | ADI | | | | |
| Example 10 | P-MAC- | A | ◯ | 23 | C |
| | ADI2 | | | | |
| Comparative | P-PHS- | A | ◯ | 33 | C |
| Example 1 | MMA | | | | |
| Comparative | XBisN-C1 | B | ◯ | 31 | A |
| Example 2 | | | | | |

As is clear from Table 1, it was confirmed that Examples 1 to 10 using a compound containing 15 to 75% by mass of iodine atoms in total or a resin having a constituent unit derived from the compound had significantly excellent sensitivity as compared with Comparative Example 1 and Comparative Example 2.

### (Preparation of Underlayer Film Composition)

Underlayer film composition solutions containing the compound of the present invention were prepared by blending the composition described in Table 2 below.

TABLE 2

| | | Thermal | | | |
|---|---|---|---|---|---|
| | | Acid | | | |
| | | Generating | | | |
| | | Agent | | | |
| | | Thermal | | Acid | |
| | | Base | | diffusion | |
| Composition | Compound | Generating | Crosslinking | controlling | |
| No. | or Resin | Agent | agent | agent | Solvent |
| Composition | BisB-2 | TAG-2689 | TMOM-BP | WPAG199 | solvent |
| B1 | 10 parts | 0.2 parts | 1 parts | 1 parts | composition A |
| | by mass | by mass | by mass | by mass | |
| Composition | BMI-1 | WPBG300 | TMOM-BP | WPAG336 | solvent |
| B2 | 10 parts | 0.2 parts | 1 parts | 1 parts | composition A |
| | by mass | by mass | by mass | by mass | |
| Composition | RXBis-N2 | TAG-2689 | TMOM-BP | WPAG367 | solvent |
| B3 | 10 parts | 0.2 parts | 1 parts | 1 parts | composition A |
| | by mass | by mass | by mass | by mass | |
| Composition | RXBis-N2 | TAG-2689 | TMOM-BP | ADCS-1 | solvent |
| B4 | 10 parts | 0.2 parts | 1 parts | 1 parts | composition A |
| | by mass | by mass | by mass | by mass | |
| Composition | I-NAFP-AL | TAG-2689 | TMOM-BP | C-1 | solvent |
| B5 | 10 parts | 0.2 parts | 1 parts | 1 parts | composition A |
| | by mass | by mass | by mass | by mass | |
| Composition | P-2I-PHS-MMA | TAG-2689 | TMOM-BP | C-2 | solvent |
| B6 | 10 parts | 0.2 parts | 1 parts | 1 parts | composition A |
| | by mass | by mass | by mass | by mass | |
| Composition | P-2I-PHS-MMA | TAG-2689 | TMOM-BP | WPBG-345 | solvent |
| B7 | 10 parts | 0.2 parts | 1 parts | 1 parts | composition A |
| | by mass | by mass | by mass | by mass | |
| Composition | XBisN-3 | TAG-2689 | TMOM-BP | WPBG-018 | solvent |
| B8 | 10 parts | 0.2 parts | 1 parts | 1 parts | composition A |
| | by mass | by mass | by mass | by mass | |
| Composition | P-MAC-ADI | TAG-2689 | TMOM-BP | WPBG-345 | solvent |
| B9 | 10 parts | 0.2 parts | 1 parts | 1 parts | composition A |
| | by mass | by mass | by mass | by mass | |
| Composition | P-MAC-ADI2 | TAG-2689 | TMOM-BP | WPBG-018 | solvent |
| B10 | 10 parts | 0.2 parts | 1 parts | 1 parts | composition A |
| | by mass | by mass | by mass | by mass | |

The components used in the preparation of the underlayer film composition solution are as follows.

### <Acid Diffusion Controlling Agent>

The following were used as acid diffusion accelerators:

WPAG199 (Bis(4-methylphenylsulfonyl)diazomehtane) (manufactured by Fujifilm Wako Pure Chemical Corporation)

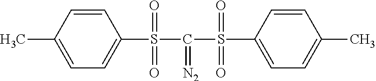

WPAG367 (Diphenyl-2,4,6-trimethylphenylsulfonium p-toluenesulfonate) (manufactured by Fujifilm Wako Pure Chemical Corporation)

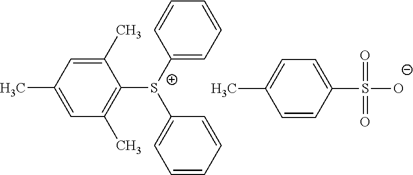 [SMILES: Cc1cc(C)c([S+](c2ccccc2)c2ccccc2)c(C)c1 ; Cc1ccc(S(=O)(=O)[O-])cc1]

WPAG336 (Diphenyl-4-methylphenylsulfonium trifluoromethanesulfonate) (manufactured by Fujifilm Wako Pure Chemical Corporation)

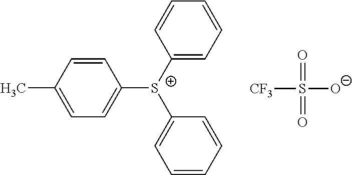 [SMILES: Cc1ccc([S+](c2ccccc2)c2ccccc2)cc1 ; O=S(=O)([O-])C(F)(F)F]

The following were used as acid diffusion inhibitors: (Compound ADCS-1)

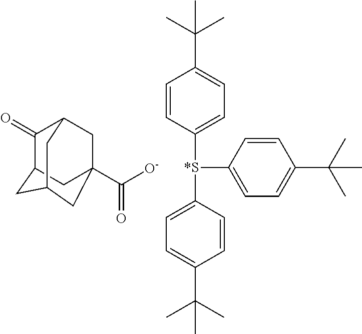

C-1

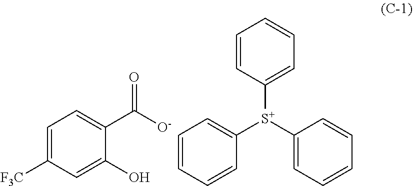 [SMILES: O=C([O-])c1ccc(C(F)(F)F)cc1O ; c1ccc([S+](c2ccccc2)c2ccccc2)cc1]

C-2

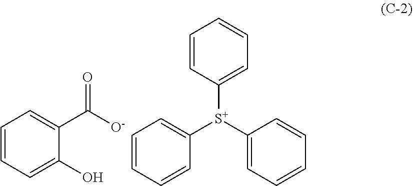 [SMILES: O=C([O-])c1ccccc1O ; c1ccc([S+](c2ccccc2)c2ccccc2)cc1]

WPBG-018 (manufactured by Fujifilm Wako Pure Chemical Corporation)

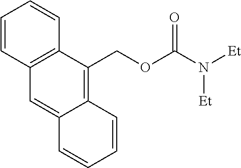 [SMILES: CCN(CC)C(=O)OCc1c2ccccc2cc2ccccc12]

WPBG-345 (manufactured by Fujifilm Wako Pure Chemical Corporation)

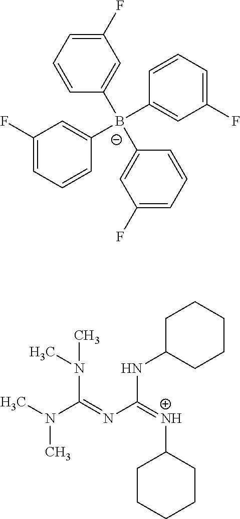 [SMILES: Fc1cccc([B-](c2cccc(F)c2)(c2cccc(F)c2)c2cccc(F)c2)c1 ; CN(C)C(=N/C(NC1CCCCC1)=[NH+]\C1CCCCC1)N(C)C]

### <Thermal Acid Generating Agent and Thermal Base Generating Agent>

WPBG300 (manufactured by Fujifilm Wako Pure Chemical Corporation)

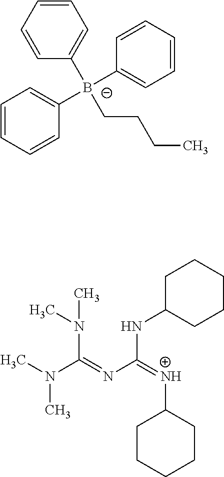

TAG-2689 (manufactured by KING CO., LTD., quaternary ammonium salt of trifluoromethanesulfonic acid)

### <Crosslinking Agent>

TMOM-BP (manufactured by Honshu Chemical Industry Co., Ltd.)

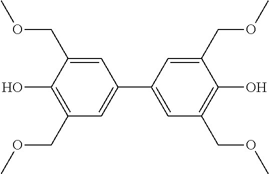

### (4) Sensitivity Evaluation

(EUV Sensitivity—n-Butyl Acetate Development)

Resist solution 2 for sensitivity evaluation and pattern evaluation was prepared by blending 5 parts by mass of the polymer MAR2 obtained in the above Synthesis Working Example of polymer for resists, 1 part by mass of triphenylsulfonium nonafluoromethanesulfonate, 0.2 part by mass of tributylamine, 80 parts by mass of PGMEA, and 12 parts by mass of PGME.

The prepared underlayer film composition was applied onto a silicon wafer and baked at 240° C. for 60 seconds to form an underlayer film having a thickness of 100 nm on the silicon wafer.

Furthermore, this underlayer film of the present invention formed on a silicon wafer was coated with the resist solution 2 and baked at 110° C. for 60 seconds to form a photoresist layer with a film thickness of 100 nm.

Then, after performing a maskless shot exposure with an increased exposure amount from 1 mJ/cm² to 80 mJ/cm² by 1 mJ/cm² at a time using an extreme ultraviolet (EUV) exposure apparatus “EUVES-7000” (product name, manufactured by Lithotech Japan Co., Ltd.), the wafer was baked at 110° C. for 90 seconds (PEB), developed with n-butyl acetate for 30 seconds, and a wafer on which a shot exposure for 80 shots was performed was obtained. The film thickness of each shot exposure area thus obtained was measured by an optical interference film thickness meter “VM 3200” (product name, manufactured by SCREEN Semiconductor Solutions Co., Ltd.), profile data of the film thickness with respect to the exposure amount was obtained, and the exposure amount at which the gradient of the film thickness variation amount with respect to the exposure amount became the largest was calculated as a sensitivity value (mJ/cm²), and used as an index of the EUV sensitivity of the resist.

### (Etch Defect Evaluation-TMAH)

The prepared underlayer film composition was applied onto a silicon wafer and baked at 240° C. for 60 seconds to form an underlayer film having a thickness 100 nm on the silicon wafer.

Furthermore, this underlayer film of the present invention formed on a silicon wafer was coated with a resist solution and baked at 110° C. for 60 seconds to form a photoresist layer with a film thickness of 100 nm.

Next, the entire wafer was shot exposed using an extreme ultraviolet (EUV) exposure apparatus “EUVES-7000” (product name, manufactured by Lithotech Japan Co., Ltd.) at an exposure amount 3% larger than the EUV sensitivity value obtained in the above EUV sensitivity evaluation in the TMAH development, the wafer was baked at 110° C. for 90 seconds (PEB), developed with 2.38% by mass tetramethylammonium hydroxide (TMAH) aqueous solution for 60 seconds, and in wafer on which the entire wafer was shot exposed 80 shots was obtained.

The prepared exposed wafer was subjected to etching treatment by an etching apparatus “Telius SCCM” (product name, manufactured by Tokyo Electron Limited) until the oxide film was etched by 60 nm using CF4/Ar gas. A wafer prepared by etching was subjected to defect evaluation by a defect inspection apparatus “Surfscan SP5” (product name, manufactured by KLA), and the number of cone defects 19 nm or larger was determined as an index of etching defects.

### (Evaluation Criteria)

A: Number of cone defects less than 20

B: 20<Number of cone defects 200

C: 200<Number of cone defects 1000

D: 1000<Number of cone defects

The obtained evaluation results are shown in Table 3.

(Etch Defect Evaluation—n-Butyl Acetate)

Etching defects were evaluated in the same manner as in the above-described etching evaluation-TMAH method except that the resist solution 2 was used as the resist solution, exposure was performed at an exposure amount 3% smaller than the EUV sensitivity in n-butyl acetate development, n-butyl acetate was used as the developer instead of the TMAH aqueous solution, and the development time was 30 seconds.

The obtained evaluation results are shown in Table 3.

TABLE 3

| | | | Sensitivity | | |
|---|---|---|---|---|---|
| | | Sensitivity | evaluation | | |
| | Underlayer film | (TMAH) | (n-butyl acetate) | Etching defect | Etching defect |
| Example No. | composition | mJ/cm2 | mJ/cm2 | TMAH | n-butyl acetate |
| Example 11 | Composition B1 | 22 | 24 | A | A |
| Example 12 | Composition B2 | 18 | 20 | A | A |
| Example 13 | Composition B3 | 21 | 22 | A | A |
| Example 14 | composition | 23 | 25 | B | B |
| | using BisB-2 | | | | |
| Example 15 | composition | 19 | 21 | B | B |
| | using BMI-1 | | | | |
| Example 16 | composition | 22 | 23 | B | B |
| | using RXBisN-2 | | | | |
| Example 17 | Composition B4 | 23 | 25 | A | A |
| Example 18 | Composition B5 | 22 | 24 | A | A |
| Example 19 | Composition B6 | 24 | 26 | A | A |
| Example 20 | Composition B7 | 24 | 26 | A | A |
| Example 21 | Composition B8 | 24 | 26 | A | A |
| Example 22 | Composition B9 | 24 | 26 | A | A |
| Example 23 | Composition B10 | 24 | 26 | A | A |
| Example 24 | composition | 22 | 24 | B | B |
| | using RXBisN-2 | | | | |
| Example 25 | composition | 21 | 23 | B | B |
| | using I-NAFP-AL | | | | |
| Example 26 | composition using | 23 | 25 | B | B |
| | P-2I-PHS-MMA | | | | |
| Example 27 | composition | 23 | 25 | B | B |
| | using XBisN-3 | | | | |
| Example 28 | composition using | 23 | 25 | B | B |
| | P-MAC-ADI | | | | |
| Example 29 | composition using | 23 | 25 | B | B |
| | P-MAC-ADI2 | | | | |

As is clear from Table 3, it was confirmed that the sensitivity can be significantly controlled by using acid diffusion controlling agents in combination.

1. A composition for lithography comprising a compound having at least one element selected from the group consisting of iodine, tellurium and fluorine or a resin having a constituent unit derived from the compound,
wherein a total mass of the elements in the compound is 15% by mass or more and 75% by mass or less.

2. The composition for lithography according to claim 1, wherein the at least one element is at least one element selected from the group consisting of iodine and tellurium.

3. The composition for lithography according to claim 1, wherein the at least one element is iodine, and a mass of the iodine in the compound is 15% by mass or more and 75% by mass or less.

4. The composition for lithography according to claim 1, wherein the compound is represented by formula (A-4a): 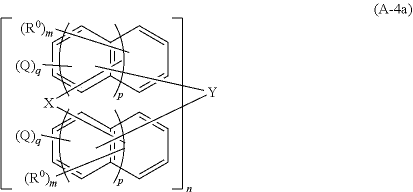
wherein
X represents an oxygen atom, a sulfur atom, a single bond, or not a crosslink;
Y is a 2n-valent group having 1 to 60 carbon atoms, or a single bond;
wherein when X is not a crosslink, Y is the 2n-valent group;
each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;
where at least one R⁰ is a hydroxy group;
each m is independently an integer of 1 to 9;
Q represents iodine, tellurium, fluorine, an alkyl group having 1 to 30 carbon atoms and containing at least iodine, tellurium or fluorine, or an aryl group having 6 to 40 carbon atoms and containing at least iodine, tellurium or fluorine;
n is an integer of 1 to 4;
each p is independently an integer of 0 to 3;
at least one of Q, R⁰ and Y contains at least one element selected from the group consisting of iodine, tellurium, and fluorine; and
each q is independently an integer of 0 to (4+2×p−m).

5. The composition for lithography according to claim 4, wherein Y is a 2n-valent hydrocarbon group having an aryl group having 6 to 60 carbon atoms and optionally having a substituent.

6. The composition for lithography according to claim 1, wherein the compound is represented by formula (A-4c): 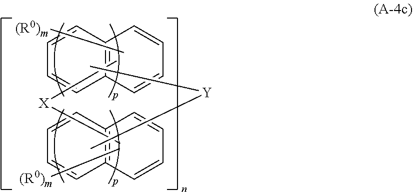
wherein
X represents an oxygen atom, a sulfur atom, a single bond, or not a crosslink;
Y is a 2n-valent group having 1 to 60 carbon atoms, or a single bond;
wherein when X is not a crosslink, Y is the 2n-valent group;
each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;
where at least one R⁰ is a hydroxy group;
at least one R⁰ is iodide or an iodine-containing group;
each m is independently an integer of 1 to 9;
n is an integer of 1 to 4; and
each p is independently an integer of 0 to 3.

7. The composition for lithography according to claim 6, wherein Y is a 2n-valent hydrocarbon group having an aryl group having 6 to 60 carbon atoms and optionally having a substituent.

8. The composition for lithography according to claim 1, wherein the compound is represented by the general formula (AM1): 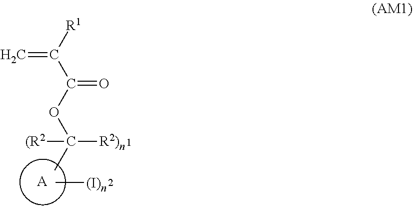
wherein
R¹ represents a hydrogen atom, methyl, or a halogen group;
each R² independently represents a hydrogen atom, a linear organic group having 1 to 20 carbon atoms, a branched organic group having 3 to 20 carbon atoms, or a cyclic organic group having 3 to 20 carbon atoms;
A represents an organic group having 1 to 30 carbon atoms;
n¹ represents 0 or 1; and
n² represents an integer of 1 to 20.

9. The composition for lithography according to claim 1, wherein the compound is represented b the neral formula (A-7): 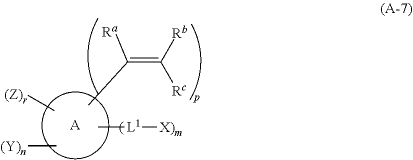
wherein
each X independently represents tellurium, I, F, or an organic group having 1 to 30 carbon atoms and having 1 or more and 5 or less substituents selected from the group consisting of tellurium, I, and F, and at least one X is tellurium or I;
L¹ represents a single bond, an ether group, an ester group, a thioether group, an amino group, a thioester group, an acetal group, a phosphine group, a phosphone group, a urethane group, a urea group, an amide group, an imide group, or a phosphate group;
m is an integer of 1 or more;
each Y independently represents a hydroxy group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group;
n is an integer of 0 or more;
each Z is independently an alkoxy group, an ester group, an acetal group, or a carbonate ester group;
r is an integer of 0 or more;
A is an organic group having 1 to 30 carbon atoms;
R^{a}, R^{b}, and R^{c} are each independently H, I, F, Cl, Br, or an organic group having 1 to 60 carbon atoms and optionally having a substituent; and
p is an integer of 1 or more.

10. The composition for lithography according to claim 1, further comprising a solvent.

11. The composition for lithography according to claim 1, further comprising an acid generating agent.

12. The composition for lithography according to claim 1, further comprising an acid diffusion accelerator.

13. The composition for lithography according to claim 1, further comprising an acid diffusion inhibitor.

14. The composition for lithography according to claim 1, further comprising a crosslinking agent.

15. The composition for lithography according to claim 1, wherein the composition is cured after formation of a thin film.

16. The composition for lithography according to claim 1, wherein the composition is for forming a resist layer contact film.

17. The composition for lithography according to claim 1, wherein the composition is for forming an underlayer film.

18. A method for forming a resist pattern, comprising:
an underlayer film formation step of forming an underlayer film on a substrate by using the composition for lithography according to claim 17;
a photoresist film formation step of forming at least one photoresist film on the underlayer film formed through the underlayer film formation step; and
a step of irradiating a predetermined region of the photoresist film formed through the photoresist film formation step with radiation for development.

19. A method for forming a circuit pattern, comprising:
an underlayer film formation step of forming an underlayer film on a substrate;
a resist layer contact film formation step of forming, by using the composition for lithography according to claim 16, a resist layer contact film on the underlayer film formed through the underlayer film formation step;
a photoresist film formation step of forming at least one photoresist film on the resist layer contact film formed through the resist layer contact film formation step;
a resist pattern formation step of irradiating a predetermined region of the photoresist film formed through the photoresist film formation step with radiation for development, thereby forming a resist pattern;
a pattern formation step of etching the resist layer contact film, or the resist layer contact film and the underlayer film with the resist pattern formed through the resist pattern formation step as a mask, thereby forming a pattern; and
a substrate pattern formation step of etching the substrate with the pattern formed through the pattern formation step as a mask, thereby forming a pattern on the substrate.

20. A compound represented by formula (A-4a): 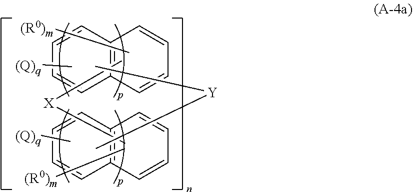
wherein
X represents an oxygen atom, a sulfur atom, a single bond, or not a crosslink;
Y is a 2n-valent group having 1 to 60 carbon atoms, or a single bond;
wherein when X is not a crosslink, Y is the 2n-valent group;
each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;
where at least one R⁰ is a hydroxy group;
each m is independently an integer of 1 to 9;
Q represents iodine, tellurium, fluorine, an alkyl group having 1 to 30 carbon atoms and containing at least iodine, tellurium or fluorine, or an aryl group having 6 to 40 carbon atoms and containing at least iodine, tellurium or fluorine;
n is an integer of 1 to 4;
each p is independently an integer of 0 to 3;
at least one of Q, R⁰ and Y contains at least one element selected from the group consisting of iodine, tellurium, and fluorine; and
each q is independently an integer of 0 to (4+2×p−m).

21. The compound according to claim 20, wherein Y is a 2n-valent hydrocarbon group having an aryl group having 6 to 60 carbon atoms and optionally having a substituent.

22. A compound represented by formula (A-4c): 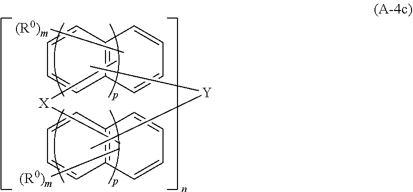
wherein
X represents an oxygen atom, a sulfur atom, a single bond, or not a crosslink;
Y is a 2n-valent group having 1 to 60 carbon atoms, or a single bond;
wherein when X is not a crosslink, Y is the 2n-valent group;
each R⁰ is independently an alkyl group having 1 to 40 carbon atoms and optionally having a substituent, an aryl group having 6 to 40 carbon atoms and optionally having a substituent, an alkenyl group having 2 to 40 carbon atoms and optionally having a substituent, an alkynyl group having 2 to 40 carbon atoms and optionally having a substituent, an alkoxy group having 1 to 40 carbon atoms and optionally having a substituent, a halogen atom, a thiol group or a hydroxy group;
where at least one R⁰ is a hydroxy group;
at least one R⁰ is iodide or an iodine-containing group;
each m is independently an integer of 1 to 9;
n is an integer of 1 to 4; and
each p is independently an integer of 0 to 3.

23. The compound according to claim 22, wherein Y is a 2n-valent hydrocarbon group having an aryl group having 6 to 60 carbon atoms and optionally having a substituent.
